(19) United States
(12) Patent Application Publication
(10) Pub. No.: US 2015/0087598 A1
(43) Pub. Date: Mar. 26, 2015

(54) TREATING MUC1-EXPRESSING CANCERS WITH HELICASE INHIBITORS

(71) Applicant: DANA-FARBER CANCER INSTITUTE, INC., Boston, MA (US)

(72) Inventor: Donald Kufe, Wellesley, MA (US)

(73) Assignee: DANA-FARBER CANCER INSTITUTE, INC., Boston, MA (US)

(21) Appl. No.: 14/397,972

(22) PCT Filed: May 13, 2013
(86) PCT No.: PCT/US2013/040772

§ 371 Date: Oct. 30, 2014

Related U.S. Application Data
(60) Provisional application No. 61/646,029, filed on May 11, 2012.

(51) Int. Cl. A61K 31/357; A61K 38/08

(52) U.S. Cl. CPC A61K 31/357; A61K 38/08
USPC 514/19.3; 514/452; 435/375

(57) ABSTRACT

The invention provides method of treating cancers that express MUC1 by the administration of eIF4A helicase inhibitors. These inhibitors may advantageously be combined with peptides that inhibit MUC1 oligomerization, or with other standard anticancer therapies such as chemo-, radio- and surgical therapies.

This application claims benefit of priority to U.S. Provisional Application Ser. No. 61/646,029, filed May 11, 2012, the entire contents of which are hereby incorporated by reference.

### BACKGROUND OF THE INVENTION

1. Field of the Invention

This invention relates to the fields of biology, medicine and oncology. In particular, the invention relates to the use of eIF4A helicase inhibitors to treat MUC1-expressing cancers.

2. Related Art

Mucin 1 (MUC1) is an oncoprotein that is aberrantly overexpressed in human cancers by mechanisms that are not clearly understood (Kufe, 2009). MUC1 consists of two subunits that form a non-covalent complex at the cell membrane (Kufe, 2009). The MUC1 N-terminal (MUC1-N) ectodomain is the mucin component of the heterodimer that contains glycosylated tandem repeats. The transmembrane MUC1 C-terminal subunit (MUC1-C) has a 58 amino acid (aa) extracellular domain that interacts with the epidermal growth factor receptor (EGFR) and other receptor tyrosines (Ramasamy et al., 2007; Kufe, 2009). Overexpression of MUC1 in transgenic mouse models is associated with binding to EGFR in mammary glands and the induction of breast tumors (Schroeder et al., 2001). The interaction between MUC1 and EGFR increases EGFR internalization and recycling at the cell membrane (Pochampalli et al., 2007). Other studies have shown that MUC1-C contributes to EGFR-mediated activation of the PI3K->AKT pathway (Raina et al., 2011). In this context, the 72 aa MUC1-C cytoplasmic domain binds to PI3K and contributes to activation of the PI3K->AKT pathway (Raina et al., 2004; Raina et al., 2011). Overexpression of the MUC1-C subunit, as found in diverse human cancers, is sufficient to induce anchorage-independent growth and tumorigenicity (Li et al., 2003; Huang et al., 2005; Kufe, 2009). Upregulation of MUC1-C also attenuates the induction of cell death in response to genotoxic, oxidative and hypoxic stress (Yin and Kufe, 2003; Ren et al., 2004; Yin et al., 2007). MUC1-C localizes to the nucleus, where it associates with transcription factors, such as NF-κB RelA and STAT3, and promotes activation of their target genes, including MUC1 itself (Ahmad et al., 2009; Ahmad et al., 2011). Thus, MUC1-C contributes, at least in part, to its own overexpression through autoinductive regulatory loops (Kufe, 2009). Based on these findings, MUC1-C has emerged as an attractive target for cancer treatment using approaches that block its function and thereby overexpression. For example, cell-penetrating peptides and small molecules that inhibit the MUC1-C cytoplasmic domain attenuate localization of MUC1-C to the nucleus of cancer cells and downregulate its overexpression (Raina et al., 2009; Joshi et al., 2009; Zhou et al., 2011). There is, however, no available information about whether MUC1-C can be targeted in cancer cells by blocking its expression at the level of translation.

### SUMMARY OF THE INVENTION

Thus, in accordance with the present invention, there is provided a method of inhibiting a cancer cell that expresses MUC1 comprising contacting said cancer cell with an inhibitor of eIF4A RNA helicase. The inhibitor may be an inhibitor of eIF4A RNA helicase expression or an inhibitor of eIF4A RNA helicase activity. The inhibitor may be silvestrol or an analog thereof. The cancer cell may be metastatic, recurrent or multidrug resistant cancer cell. The method may further comprise contacting said cancer cell with said inhibitor more than once. The cancer cell may be a carcinoma cell, a leukemia cell or a myeloma cell. The carcinoma cell may be a prostate or breast carcinoma cell.

The method may further comprise contacting said cancer cell with a MUC1 peptide of at least 4 consecutive MUC1 residues and no more than 20 consecutive MUC1 residues and comprising the sequence CQC, wherein the amino-terminal cysteine of CQC is covered on its NH₂-terminus by at least one amino acid residue that need not correspond to the native MUC1 transmembrane sequence. The peptide may comprises at least 6, 7 or 8 consecutive MUC1 residues, comprising CQCRRK (SEQ ID NO:4). The peptide may contain no more than 10 consecutive residues, 11 consecutive residues, 12 consecutive residues, 13 consecutive residues, 14 consecutive residues, 15 consecutive residues, 16 consecutive residues, 17 consecutive residues, 18 consecutive residues or 19 consecutive residues of MUC1. The peptide may be fused to a cell delivery domain, such as poly-D-R, poly-D-P or poly-D-K. The peptide may comprises all L amino acids or all D amino acids, or a mix of L and D amino acids.

In another embodiment, there is provided a method of treating MUC1-expressing cancer in a subject comprising administering to said subject an inhibitor of eIF4A RNA helicase. The inhibitor may be an inhibitor of eIF4A RNA helicase expression or an inhibitor of eIF4A RNA helicase activity. The inhibitor may be silvestrol or an analog thereof. The cancer may be metastatic, recurrent or multidrug resistant. The method may further comprise administering said inhibitor more than once. The cancer may be a carcinoma, a leukemia or a myeloma. The carcinoma may be a prostate or breast carcinoma. The method may further comprise, prior to administering, the step of assessing MUC1 expression in a cancer cell from said subject. The assessing may comprise MUC1 nucleic acid detection or MUC1 protein detection.

The method may further comprise administering to said subject a second anti-cancer therapy. The second anti-cancer therapy may be surgery, chemotherapy, radiotherapy, hormonal therapy, toxin therapy, immunotherapy, cryotherapy, a MUC1 ligand TRAP, or a small molecule inhibiting MUC1 dimer formation. The second anti-cancer therapy is administered prior to said inhibitor, after said inhibitor, or at the same time as said inhibitor. The second anti-cancer therapy may comprise administering to said subject a MUC1 peptide of at least 4 consecutive MUC1 residues and no more than 20 consecutive MUC1 residues and comprising the sequence CQC, wherein the amino-terminal cysteine of CQC is covered on its NH₂-terminus by at least one amino acid residue that need not correspond to the native MUC1 transmembrane sequence. The peptide may comprise at least 6, 7, 8, 9, 10, 11 or 12 consecutive MUC1 residues, comprising CQCRRK (SEQ ID NO:4). The peptide may contain no more than 10 consecutive residues, 11 consecutive residues, 12 consecutive residues, 13 consecutive residues, 14 consecutive residues, 15 consecutive residues, 16 consecutive residues, 17 consecutive residues, 18 consecutive residues or 19 consecutive residues of MUC1. The peptide may be fused to a cell delivery domain, such as poly-D-R, poly-D-P or poly-D-K. The peptide may comprise all L amino acids or all D amino acids or a mix of L and D amino acids.

The administering may comprise intravenous, intra-arterial, intra-tumoral, subcutaneous, topical or intraperitoneal administration. The administering may also comprise local, regional (e.g., into tumor vasculature), systemic, or continual administration. Inhibiting may comprise inducing growth arrest of said tumor cell, apoptosis of said tumor cell and/or necrosis of a tumor tissue comprising said tumor cell. The subject may be human. The inhibitor may be administered at 0.1-500 mg/kg/d, or at 10-100 mg/kg/d. The inhibitor may be administered daily, such as daily for 7 days, 2 weeks, 3 weeks, 4 weeks, one month, 6 weeks, 8 weeks, two months, 12 weeks, or 3 months. The inhibitor may be administered weekly, such as weekly for 2 weeks, 3 weeks, 4 weeks, 6 weeks, 8 weeks, 10 weeks, or 12 weeks.

Also provided are (i) a kit comprising (a) a MUC1 detection agent and (b) and eIF4A inhibitor, and (ii) a use of an eIF4A inhibitor in the treatment of MUC1-expressing cancer.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed.

FIGS. 1A-D. Stimulation of non-malignant MCF-10A breast epithelial cells with EGF or HRG induces MUC1 expression. (FIG. 1A) Lysates from MCF-10A cells and the indicated breast cancer cells were immunoblotted with anti-MUC1-C and anti-β-actin. (FIG. 1B) MCF-10A cells were stimulated with 100 ng/ml EGF for the indicated times. Lysates were immunoblotted with anti-MUC1-C and anti-β-actin (left). Intensity of the MUC1-C signals was determined by densitometric scanning. The results (mean±SD of three replicates) are expressed as relative MUC1-C levels compared to that obtained for the untreated control (assigned a value of 1) (right). (FIG. 1C) MCF-10A cells were stimulated with 10 ng/ml HRG for the indicated times. Lysates were immunoblotted with the indicated antibodies. (FIG. 1D) MCF-7 cells were stimulated 100 ng/ml EGF for the indicated times. Lysates were immunoblotted with the indicated antibodies (left). The results (mean±SD of three replicates) are expressed as relative MUC1-C levels compared to that obtained for the untreated control (assigned a value of 1) (right).

FIGS. 2A-D. Activation of the PI3K->AKT pathway induces MUC1-C translation. (FIG. 2A) MCF-10A cells were transfected with the control pGL3 (CTL) or pMUC1-Luc in the presence of Renilla plasmid for 24 h. The cells were then stimulated with EGF for 5 h and then assayed for luciferase activity. The results are expressed as the fold-activation (mean±SD of three determinations) relative to that obtained for the pGL3 plasmid (left). MCF-10A cells were stimulated with EGF for the indicated times. MUC1 mRNA levels (mean±SD of three determinations) were assayed by qRT-PCR (right). (FIG. 2B) MCF-10A cells were stimulated with EGF in the absence and presence of 10 ng/ml CHX for the indicated times. Lysates were immunoblotted with anti-MUC1-C and anti-β-actin. (FIG. 2C) MCF-10A cells were stimulated with EGF in the presence of 10 μM U0126 or 50 μM LY294002 for 8 and 24 h. Lysates were immunoblotted with the indicated antibodies (left). The results (mean±SD of three replicates) are expressed as relative MUC1-C levels compared to that obtained for the untreated control (assigned a value of 1) (right). (FIG. 2D) MCF-10A cells were stimulated with EGF in the presence of 250 or 500 nM BEZ235. Lysates were immunoblotted with the indicated antibodies (left). The results (mean±SD of three replicates) are expressed as relative MUC1-C levels compared to that obtained for the untreated control (assigned a value of 1) (right).

FIGS. 3A-D. Growth factor-induced MUC1-C expression is regulated by cap-dependent translation. (FIG. 3A) MCF-10A cells were stimulated with EGF in the presence of 250 or 500 nM BEZ235 for the indicated times. Lysates were immunoblotted with the indicated antibodies (left). The results (mean±SD of three replicates) are expressed as relative MUC1-C levels compared to that obtained for the untreated control (assigned a value of 1) (right). (FIG. 3B) MCF-10A cells were stimulated with EGF in the presence of 100 nM rapamycin for the indicated times. Lysates were immunoblotted with anti-MUC1-C and anti-3-actin (left). The results (mean±SD of three replicates) are expressed as relative MUC1-C levels compared to that obtained for the untreated control (assigned a value of 1) (right). (FIG. 3C) MCF-10A cells were transfected to express a control siRNA or a S6K1 siRNA pool. Lysates from the transfected cells were immunoblotted with the indicated antibodies. (FIG. 3D) MCF-10A cells transfected with the CsiRNA or S6K1siRNA were left untreated (CTL) or stimulated with EGF for 24 h. Lysates were immunoblotted with the indicated antibodies (left). The results (mean±SD of three replicates) are expressed as relative MUC1-C levels compared to that obtained for the control (assigned a value of 1) (right).

FIGS. 4A-E. MUC1-C contributes to EGFR-mediated signaling and cell growth. (FIG. 4A) MCF-10A cells were stimulated with EGF for 8 and 24 h. Lysates were immunoblotted with the indicated antibodies (left). The results (mean±SD of three replicates) are expressed as relative PDCD4 levels compared to that obtained for the untreated control (assigned a value of 1) (right). (FIG. 4B) Lysates from MCF-10A, MCF-7, BT-549 and MDA-MB-468 cells were immunoblotted with the indicated antibodies (left). The results (mean±SD of three replicates) are expressed as relative PDCD4 levels compared to that obtained for MCF-10A cells (assigned a value of 1) (right). (FIGS. 4C-D) MCF-10A cells were left untreated (CTL) and stimulated with EGF (FIG. 4C) or HRG (FIG. 4D) in the presence of the indicated concentrations of silvestrol for 24 h. Lysates were immunoblotted with anti-MUC1-C and anti-β-actin (left). The results (mean±SD of three replicates) are expressed as relative MUC1-C levels compared to that obtained for the control (assigned a value of 1) (right). (FIG. 4E) MCF-10A cells were treated with 100 nM silvestrol for the indicated times. Lysates were immunoblotted with anti-PDCD4 and anti-β-actin.

FIGS. 5A-F. MUC1-C translation is regulated by PI3K->AKT signaling and eIF4A in breast cancer cells. (FIG. 5A) MCF-10A cells were stimulated with EGF for the indicated times. Lysates were precipitated with a control IgG or anti-EGFR. The precipitates were immunoblotted with anti-MUC1-C or anti-EGFR. (FIG. 5B) MCF-10A cells were left untreated (CTL) or stimulated with EGF for 24 h. Cells were stained with anti-MUC1-C and anti-EGFR, and analyzed by confocal microscopy (left). The images were analyzed by Image J (32) to confirm increased colocalization of EGFR and MUC1-C in the response to EGF stimulation (right). (FIG. 5C) Lysates from MCF-10A cells stably transfected to express a control siRNA or a MUC1 siRNA were immunoblotted with the indicated antibodies (left). The MCF-10A/CsiRNA and MCF-10A/MUC1siRNA cells were left untreated or stimulated with EGF for 24 h (right). Lysates were immunblotted with the indicated antibodies. (FIG. 5D) MCF-10A/CsiRNA and MCF-10A/MUC1siRNA cells were stimulated with EGF for 24 h. Control (CTL) and EGF-treated cells were stained with PI and analyzed for cell cycle distribution by flow cytometry. The percentage of cells in G1, S and G2 phase are included in the panels. (FIG. 5E) MCF-10A/CsiRNA and MCF-10A/MUC1siRNA cells were stimulated with EGF for 24 h, reseeded and then counted at 48 h. The results are expressed as cell number (mean±SD of three determinations). (FIG. 5F) MCF-10A/CsiRNA and MCF-10A/MUC1siRNA cells were stimulated with EGF for 24 h and reseeded into 6-well plates (1000 cells per well). Colonies were stained with crystal violet, photographed (left) and counted (right) on day 7. The results are expressed as colony number (mean±SD of three determinations) (right).

FIGS. 6A-D. MUC1-C translation is inhibited by CR-1-31-B in MCF-10A cells. (FIG. 6A) Structures of the indicated compounds. ((FIGS. 6B and 6C) MCF-10A cells were left untreated (CTL) and stimulated with EGF ((FIG. 6B) or HRG ((FIG. 6C) in the presence of the indicated concentrations of CR-1-31-B (left) or inactive CR-1-30-B (right) for 24 h. (FIG. 6D) MCF-10A cells were stimulated with EGF in the absence (CTL) or presence of 100 nM CR-1-31-B or CR-1-30-B for 24 h, reseeded and then counted at 48 h. Viable cell number (mean±SD of three determinations) was determined by trypan blue exclusion.

FIGS. 7A-F. Downregulation of MUC1-C expression by CR-1-31-B in breast cancer cells. (FIGS. 7A and 7B) MCF-7 (FIG. 7A) and BT-549 (FIG. 7B) cells were treated with LY294002 for the indicated times. Lysates were immunoblotted with the indicated antibodies. (FIG. 7C-D) MCF-7 (FIG. 7C) and MDA MB-468 (FIG. 7D) cells were treated with 10 or 100 nM silvestrol for the indicated times. Lysates were immunoblotted with anti-MUC1-C and anti-β-actin. (FIG. 7E-F) MCF-7 (FIG. 7E) and MDA-MB-468 (FIG. 7F) cells were treated with 100 nM CR-1-31-B or inactive CR-1-30-B for the indicated times. Lysates were immunoblotted with anti-MUC1-C and anti-β-actin.

FIG. 8. Proposed autoinductive loop in which MUC1-C contributes to activation of the EGFR->PI3K->AKT->mTOR pathway and thereby increased translation of the MUC1-C protein. MUC1-C forms complexes with EGFR at the cell membrane that are mediated by extracellular galectin-3 bridges. Stimulation of EGFR with EGF induces phosphorylation of the MUC1-C cytoplasmic domain, promotes binding of the PI3K SH2 domains and thereby activation of the PI3K->AKT->mTOR pathway. mTOR-mediated phosphorylation and activation of S6K1 induces degradation of PDCD4, an inhibitor of the eIF4A RNA helicase. Derepression of eIF4A activity stimulates MUC1-C translation with marked increases in MUC1-C protein and, in turn, the formation of EGFR/MUC1-C complexes. This autoinductive loop is constitutively activated in breast cancer cells and disrupted by the eIF4A inhibitors, silvestrol and CR-1-31-B.

### DESCRIPTION OF ILLUSTRATIVE EMBODIMENTS

As discussed above, overexpression of the MUC1-C subunit, as found in diverse human cancers, is sufficient to induce anchorage-independent growth and tumorigenicity (Li et al., 2003; Huang et al., 2005; Kufe, 2009). Upregulation of MUC1-C also attenuates the induction of cell death in response to genotoxic, oxidative and hypoxic stress (Yin and Kufe, 2003; Ren et al., 2004; Yin et al., 2007). The 72 aa MUC1-C cytoplasmic domain has been shown to bind to PI3K and contribute to activation of the PI3K->AKT pathway (Raina et al., 2004; Raina et al., 2011). MUC1-C also localizes to the nucleus, where it associates with transcription factors, such as NF-κB RelA and STAT3, and promotes activation of their target genes, including MUC1 itself (Ahmad et al., 2009; Ahmad et al., 2011). Thus, MUC1-C contributes, at least in part, to its own overexpression through autoinductive regulatory loops (Kufe, 2009).

Here, the inventor shows that growth factor stimulation of non-malignant MCF-10A breast epithelial cells is associated with activation of the PI3K->AKT->mTORC1 pathway and thereby induction of MUC1-C translation. In concert with involvement of the eIF4A RNA helicase, growth factor-induced MUC1-C translation in MCF-10A cells was inhibited by silvestrol and another eIF4A inhibitor, designated CR-1-31-B. The results also show that treatment of human breast cancer cells with eIF4A inhibitors is associated with downregulation of MUC1-C expression. As such, impairing eIF4A activity presents an attractive therapeutic avenue for the treatment of MUC1-involved cancers. These and other aspects of the invention are described in greater detail below.

### I. MUC1

A. Structure

MUC1 is a mucin-type glycoprotein that is expressed on the apical borders of normal secretory epithelial cells (Kufe et al., 1984). MUC1 forms a heterodimer following synthesis as a single polypeptide and cleavage of the precursor into two subunits in the endoplasmic reticulum (Ligtenberg et al., 1992). The cleavage may be mediated by an autocatalytic process (Levitan et al., 2005). The >250 kDa MUC1 N-terminal (MUC1 N-ter, MUC1-N) subunit contains variable numbers of 20 amino acid tandem repeats that are imperfect with highly conserved variations and are modified by O-linked glycans (Gendler et al., 1988; Siddiqui et al., 1988). MUC1-N is tethered to the cell surface by dimerization with the ˜23 kDa C-terminal subunit (MUC1 C-ter, MUC1-C), which includes a 58 amino acid extracellular region, a 28 amino acid transmembrane domain and a 72 amino acid cytoplasmic domain (CD; SEQ ID NO:1) (Merlo et al., 1989). The human MUC1 sequence is shown below:
GSVVVQLTLAFREGTINVHDVETQFNQYKTEAASRYNLTISDVSVSDVPFPFS AQSGAGVPGWGIALLVLVCVLVALAIVYLIALAVCQCRRKNYGQLDIFPAR DTYHPMSEYPTYHTHGRYVPPSSTDRSPYEKVSAGNGGSSLSYTNPAVAA TSANL (SEQ ID NO:2)

The bold sequence indicates the CD, and the underlined portion is an oligomer-inhibiting peptide (SEQ ID NO:3). With transformation of normal epithelia to carcinomas, MUC1 is aberrantly overexpressed in the cytosol and over the entire cell membrane (Kufe et al., 1984; Perey et al., 1992). Cell membrane-associated MUC1 is targeted to endosomes by clathrin-mediated endocytosis (Kinlough et al., 2004). In addition, MUC1-C, but not MUC1-N, is targeted to the nucleus (Baldus et al., 2004; Huang et al., 2003; Li et al., 2003a; Li et al., 2003b; Li et al., 2003c; Wei et al., 2005; Wen et al., 2003) and mitochondria (Ren et al., 2004).

B. Function

MUC1 interacts with members of the ErbB receptor family (Li et al., 2001b; Li et al., 2003c; Schroeder et al., 2001) and with the Wnt effector, β-catenin (Yamamoto et al., 1997). The epidermal growth factor receptor and c-Src phosphorylate the MUC1 cytoplasmic domain (MUC1-CD) on Y-46 and thereby increase binding of MUC1 and β-catenin (Li et al., 2001a; Li et al., 2001b). Binding of MUC1 and β-catenin is also regulated by glycogen synthase kinase 313 and protein kinase Cδ (Li et al., 1998; Ren et al., 2002). MUC1 colocalizes with β-catenin in the nucleus (Baldus et al., 2004; Li et al., 2003a; Li et al., 2003c; Wen et al., 2003) and coactivates transcription of Wnt target genes (Huang et al., 2003). Other studies have shown that MUC1 also binds directly to p53 and regulates transcription of p53 target genes (Wei et al., 2005). Notably, overexpression of MUC1 is sufficient to induce anchorage-independent growth and tumorigenicity (Huang et al., 2003; Li et al., 2003b; Ren et al., 2002; Schroeder et al., 2004).

Most mitochondrial proteins are encoded in the nucleus and are imported into mitochondria by translocation complexes in the outer and inner mitochondrial membranes. Certain mitochondrial proteins contain N-terminal mitochondrial targeting sequences and interact with Tom20 in the outer mitochondrial membrane (Truscott et al., 2003). Other mitochondrial proteins contain internal targeting sequences and interact with the Tom70 receptor (Truscott et al., 2003). Recent work showed that mitochondrial proteins without internal targeting sequences are delivered to Tom70 by a complex of HSP70 and HSP90 (Young et al., 2003).

### II. EIF4A HELICASE

A. Helicases

The eukaryotic initiation factor-4A (eIF4A) family consists of 3 closely related proteins EIF4A1, EIF4A2, and EIF4A3. These factors are required for the binding of mRNA to 40S ribosomal subunits. In addition these proteins are helicases that function to unwind double-stranded RNA. The mechanisms governing the basic subsistence of eukaryotic cells are immensely complex; it is unsurprising, therefore, that regulation occurs at a number of stages of protein synthesis—the regulation of translation has become a well-studied field. Human translational control is of increasing research interest as it has connotations in a range of diseases. Orthologs of many of the factors involved in human translation are shared by a range of eukaryotic organisms; some of which are used as model systems for the investigation of translation initiation, for example: sea urchin eggs and rabbit reticulocytes. Monod and Jacob were among the first to propose that “the synthesis of individual proteins may be provoked or suppressed within a cell, under the influence of specific external agents, and the relative rates at which different proteins may be profoundly altered, depending upon external conditions.” Almost half a century after the flurry of postulations arising from the revelation of the central dogma of molecular biology, of which the preceding supposition by Monod and Jacob is an example; contemporary researchers still have much to learn about the modulation of genetic expression. Synthesis of protein from mature messenger RNA in eukaryotes is divided into translation initiation, elongation, and termination of these stages; the initiation of translation is the rate limiting step. Within the process of translation initiation; the bottleneck occurs shortly before the ribosome binds to the 5′ m7GTP facilitated by a number of proteins; it is at this stage that constrictions born of stress, amino acid starvation etc take effect.

Eukaryotic initiation factor (eIF) complex 2 forms a ternary complex with GTP and the initiator Met-tRNA—this process is regulated by guanine nucleotide exchange and phosphorylation and serves as the main regulatory element of the bottleneck of protein expression. Before translation can progress to the elongation stage, a number of initiation factors must facilitate the synergy of the ribosome and the mRNA and ensure that the 5′ UTR of the mRNA is sufficiently devoid of secondary structure. Binding in this way is facilitated by group 4 eukaryotic initiation factors; eIF4 has implications in the normal regulation of translation as well as the transformation and progression of cancerous cells; as such, it represents an interesting field of research.

The repertoire of compounds involved in eukaryotic translation consists of initiation factor classes 1-6; eIF4 is responsible for the binding of capped mRNA to the 40S ribosomal subunit via eIF3. The mRNA cap is bound by eIF4E (25 kDa), eIF4G (185 kDa) acts as a scaffold for the complex whilst the ATP-dependent RNA helicase eIF4A (46 kDa) processes the secondary structure of the mRNA 5′ UTR to render it more conducive to ribosomal binding and subsequent translation. Together these three proteins are referred to as eIF4F. For maximal activity; eIF4A also requires eIF4B (80 kDa), which itself is enhanced by eIF4H (25 kDa). A study conducted by Bi et al. into wheat germ seemed to indicate that eIF4A has a higher binding affinity for ADP than ATP except in the presence of eIF4B, which increased the ATP binding affinity tenfold without affecting ADP affinity. Once bound to the 5′ cap of mRNA, this 48S complex then searches for the (usually) AUG start codon and translation begins.

In humans, the gene encoding eIF4A isoform I has a transcript length of 1741 bp, contains 11 exons, and is located on chromosome 17. The genes for human isoforms II and III reside on chromosomes 3 and 17 respectively. The 407 residue, 46 kDa, protein eIF4A is the prototypical member of the DEAD box helicase family, so-called due to their conserved four-residue D-E-A-D sequence. This family of helicases is found in a range of prokaryotic and eukaryotic organisms including humans, wherein they catalyse a variety of processes including embryogenesis and RNA splicing as well as translation initiation. Crystallographic analysis of yeast eIF4A carried out by Carruthers et al. (2000) revealed that the molecule is approximately 80 Å in length and has a “dumbbell” shape where the proximal section represents an 11 residue (18 Å) linker postulated to confer a degree of flexibility and distension to the molecule in solution. eIF4A is an abundant cytoplasmic protein.

Three isoforms of eIF4A exist; I and II share 95% amino acid similarity and have been found simultaneously in rabbit reticulocyte eIF4F in a ratio of 4:1, respectively. The third isoform; eIF4A III, which shares only 65% similarity to the other isoforms is believed to be a core component of the exon junction complex involved in pre-mRNA splicing.

B. Function and Inhibition

As stated above, protein synthesis is a tightly regulated process that is limited by translation initiation, a step controlled by the eIF4F complex at the level of ribosomal recruitment (Sonenberg and Hinnebusch, 2009). The eIF4F complex is formed by binding of eIF4E to the 5′ cap structure of mRNAs and thereby recruitment of eIF4G and eIF4A. Overexpression of eIF4E has been documented in diverse human cancers and linked to transformation (De Benedetti and Graff, 2004). eIF4E contributes to the malignant phenotype by selectively promoting the translation of certain oncoproteins, such as cyclin D1, MYC and MCL1, that are involved in growth and survival (De Benedetti and Graff, 2004; Wendel et al., 2007). The PI3K->AKT pathway is a major regulator of protein synthesis that is upstream to the mammalian target of rapamycin complex 1 (mTORC1) (Ma and Blenis, 2009). mTORC1 regulates eIF4E activity by phosphorylation and thereby inactivation of the inhibitory eIF4E binding proteins (4E-BPs). mTORC1 also contributes to cap-dependent translation by activating 40S ribosomal protein S6 kinases (S6Ks) that, in turn, enhance the eIF4A RNA helicase activity (Sonenberg and Hinnebusch, 2009). S6K induces degradation of the tumor suppressor programmed cell death protein 4 (PDCD4), which is an eIF4A inhibitor (Dorrello et al., 2006). eIF4A initiates translation by unwinding highly structured 5′ untranslated regions (UTRs) in mRNAs, such as those encoding cyclin D1 and MYC (Rogers et al., 2002). In this way, cancer cells can modulate translation in response to growth signals through mTORC1-induced (i) binding of eIF4E to the 5′ cap structure and (ii) activation of the eIF4A RNA helicase function. Dysregulation of translation in malignant cells has supported the development of agents that target eIF4E (Blagden and Willis, 2011) and eIF4A (Bordeleau et al., 2008; Lucas et al., 2009). For example, the natural product silvestrol is a potent inhibitor of the eIF4A RNA helicase function that blocks cap-dependent translation and decreases production of cyclin D1, MYC and MCL1 (Lucas et al., 2009; Schatz et al., 2011). Silvestrol has also been shown to be active against cancer cells growing in vitro and in animal models (Lucas et al., 2009; Bordeleau et al., 2008; Schatz et al., 2011). These findings have indicated that constitutive activation of PI3K->AKT->mTORC1 signaling in cancer cells can be blocked in part by targeting downstream effectors of translation.

### III. HELICASE INHIBITORS

The present invention contemplates the use of a variety of different inhibitors for treatment of MUC1-expressing cancers. One class of agents that can be used are chemical/small molecule inhibitors. These are well-known in the art and can be exemplified, in one embodiment, by silvestrol. U.S. Pat. No. 6,710,075 describes silvestrol and related cyclopenta[b]benzofuran compounds carrying a sterically bulky group at the 6-oxy-position, in particular, a dioxanyl group. This dioxanyl group has not previously been reported from a natural source. It is believed that the presence at the 6-oxy-position of a sterically bulky group, i.e., spatially larger than a methoxy group, may confer both cytotoxic and cytostatic properties on the compounds having a cyclopenta[b]benzofuran core. Rodrigo et al. (2012) have described activity of rocaglates/rocaglamides, a class of natural products known to display potent anticancer activity. They recently reported synthesis of various rocaglamide analogues and identification of a hydroxamate derivative (−)-9 having activity similar to silvestrol in vitro and ex vivo for inhibition of protein synthesis. They also showed that (−)-9 synergizes with doxorubicin in vivo to reduce Eμ-Myc driven lymphomas. Related compounds are disclosed in U.S. Pat. Nos. 6,420,393 and 6,943,182.

Alternative inhibitors include biological molecules, such as antisense and siRNA constructs. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

### IV. THERAPIES

A. Pharmaceutical Formulations and Routes of Administration Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Ed., 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Cancers

Oncogenesis is a multistep biological process, which is presently known to occur by the accumulation of genetic damage. On a molecular level, the process of tumorigenesis involves the disruption of both positive and negative regulatory effectors (Weinberg, 1989). The molecular basis for human carcinomas has been postulated to involve a number of oncogenes, tumor suppressor genes and repair genes. As discussed above, MUC1 has been identified as a major participant in aberrant signaling in abnormal cells, leading to cancer.

The present invention involves the treatment of cancer, in particular, those expressing MUC1. Thus, it is contemplated that a wide variety of tumors may be treated according to the present invention, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree—indeed, any increase in patient comfort, function or longevity may be considered a successful treatment. Of course, it may be that the tumor growth is completely blocked or that some tumor regression is achieved. Clinical terminology such as “remission,” “surgically resectable” and “reduction of tumor” burden also are contemplated given their normal usage.

C. Treatment Methods

eIF4A inhibitors can be administered to mammalian subjects (e.g., human patients) alone or in conjunction with other drugs that modulate inflammation. The compounds can also be administered to subjects that are genetically and/or due to, for example, physiological and/or environmental factors, or susceptible to cancer, e.g., subjects with a family history of cancer.

The dosage required depends on the choice of the route of administration; the nature of the formulation; the nature of the patient's disease; the subject's size, weight, surface area, age, and sex; other drugs being administered; and the judgment of the attending physician. Suitable dosages are in the range of 0.0001-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more times). Encapsulation of the polypeptide in a suitable delivery vehicle (e.g., polymeric microparticles or implantable devices) may increase the efficiency of delivery, particularly for oral delivery.

D. Combination Therapies

It is common in many fields of medicine to treat a disease with multiple therapeutic modalities, often called “combination therapies.” Cancers are no exception.

To treat cancers using the methods and compositions of the present invention, one would generally contact a target cell or subject with a eIF4A inhibitor and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the eIF4A inhibitor and the other includes the other agent.

Alternatively, the eIF4A inhibitor may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the eIF4A inhibitor or the other therapy will be desired. Various combinations may be employed, where the eIF4A inhibitor is “A,” and the other therapy is “B,” as exemplified below:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

Other combinations are contemplated, as discussed below.

Administration of the therapy or agents to a patient will follow general protocols for the treatment/administration of such compounds, taking into account the toxicity, if any, of the therapy. It is expected that the treatment cycles would be repeated as necessary. It also is contemplated that various standard cancer therapies, as well as surgical intervention, may be applied in combination with the described therapy.

The terms “contacted” and “exposed,” when applied to a cell, are used herein to describe the process by which a eIF4A inhibitor and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

1. Chemotherapeutics

eIF4A inhibitor therapies may be combined, advantageously, with conventional cancer therapies. These include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

Suitable therapeutic agents include, for example, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

In some embodiments, the chemotherapeutic agent is any of (and in some embodiments selected from the group consisting of) adriamycin, colchicine, cyclophosphamide, actinomycin, bleomycin, daunorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Herceptin®, vinorelbine, Doxil®, capecitabine, Gleevec®, Alimta®, Avastin®, Velcade®, Tarceva®, Neulasta®, Lapatinib, STI-571, ZD1839, Iressa® (gefitinib), SH268, genistein, CEP2563, SU6668, SU11248, EMD121974, and Sorafenib.

In some embodiments, the chemotherapeutic agent is a composition comprising nanoparticles comprising a thiocolchicine derivative and a carrier protein (such as albumin)

In further embodiments, a combination of chemotherapeutic agents is administered to prostate cancer cells. The chemotherapeutic agents may be administered serially (within minutes, hours, or days of each other) or in parallel; they also may be administered to the patient in a pre-mixed single composition. The composition may or may not contain a glucocorticoid receptor antagonist. Combinations of prostate cancer therapeutics include, but are not limited to the following: AT (Adriamycin and Taxotere), AC±T: (Adriamycin and Cytoxan, with or without Taxol or Taxotere), CMF (Cytoxan, methotrexate, and fluorouracil), CEF (Cytoxan, Ellence, and fluorouracil), FAC (fluorouracil, Adriamycin, and Cytoxan), CAF (Cytoxan, Adriamycin, and fluorouracil) (the FAC and CAF regimens use the same medicines but use different doses and frequencies), TAC (Taxotere, Adriamycin, and Cytoxan), and GET (Gemzar, Ellence, and Taxol). In some embodiments trastuzumab (Herceptin®) is administered to a prostate cancer patient with a glucocorticoid receptor antagonist, which may be with or without a chemotherapeutic or a combination of chemotherapeutics.

The term “a serine/threonine kinase inhibitor,” as used herein, relates to a compound which inhibits serine/threonine kinases. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-κB, eIF2α, COX2, RANTES, IL8,CYP2A5, IGF-1, CYP2B1, CYP2B2, CYP2H1, ALAS-1, HIF-1, erythropoietin and/or CYP1A1. An example of a serine/theronine kinase inhibitor includes, but is not limited to, Sorafenib and 2-aminopurine, also known as 1H-purin-2-amine(9CI). Sorafenib is marketed as NEXAVAR. The compounds can be used in combination with a glucocorticoid receptor antagonist.

The term “an angiogenesis inhibitor,” as used herein, relates to a compound which targets, decreases or inhibits the production of new blood vessels. Targets of an angiogenesis inhibitor include, but are not limited to, methionine aminopeptidase-2 (MetAP-2), macrophage inflammatory protein-1 (MIP-1α), CCL5, TGF-β, lipoxygenase, cyclooxygenase, and topoisomerase. Indirect targets of an angiogenesis inhibitor include, but are not limited to, p21, p53, CDK2 and collagen synthesis. Examples of an angiogenesis inhibitor include, but are not limited to, Fumagillin, which is known as 2,4,6,8-decatetraenedioic acid, mono[3R,4S,5S,6R)-5-methoxy-4-[(2R,3R)-2-methyl-3-(3-methyl-2-butenyl)oxi-rany]-1-oxaspiro[2.5]oct-6-yl]ester, (2E,4E,6E,8E)-(9CI); Shikonin, which is also known as 1,4-naphthalenedione, 5,8-dihydroxy-2-[(1R)-1-hydroxy-4-methyl-3-pentenyl]-(9CI); Tranilast, which is also known as benzoic acid, 2-[[3-(3,4-dimethoxyphenyl)-1-oxo-2-propenyl]amino]-(9CI); ursolic acid; suramin; thalidomide and lenalidomide, and marketed as REVLIMID. The compounds can be used in combination with a glucocorticoid receptor antagonist.

2. Radiation

Radiation therapy that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems. Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

3. Gene Therapy

Gene therapy is the insertion of polynucleotides, including DNA or RNA, into an individual's cells and tissues to treat a disease. Antisense therapy is also a form of gene therapy in the present invention. A therapeutic polynucleotide may be administered before, after, or at the same time of a first cancer therapy. Delivery of a vector encoding a variety of proteins is encompassed within the invention. For example, cellular expression of the exogenous tumor suppressor oncogenes would exert their function to inhibit excessive cellular proliferation, such as p53, p16, FHIT and C-CAM.

4. Other Agents

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue. Upon excision of part of all of cancerous cells, tissue, or tumor, a cavity may be formed in the body. Treatment may be accomplished by perfusion, direct injection or local application of the area with or without an additional anti-cancer therapy.

6. MUC1 Peptides

The present invention contemplates the use of MUC1 peptides as therapeutics in combination with the aforementioned eIF4A inhibitors. The structural features of these peptides are as follows. First, the peptides have no more than 20 consecutive residues of MUC1. Thus, the term “a peptide having no more than 20 consecutive residues,” even when including the term “comprising,” cannot be understood to comprise a greater number of consecutive MUC1 residues. Second, the peptides will contain the CQC motif, and may further comprise the CQCR, CQCRR, or CQCRRK motifs. Thus, the peptides will have, at a minimum, these four, five or six consecutive residues of the MUC1-C domain. Third, the peptides will have at least one amino acid residue attached to the NH₂-terminal side of the first C residue in the CQCRRK motif, such that the first C residue is “covered” by that at least one amino acid attached thereto. This residue may be native to MUC1 (i.e., from the transmembrane domain), may be selected at random (any of the twenty naturally-occurring amino acids or analogs thereof), or may be part of another peptide sequence (e.g., a tag sequence for purification, a stabilizing sequence, or a cell delivery domain).

In general, the peptides will be 50 residues or less, again, comprising no more than 20 consecutive residues of MUC1. The overall length may be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues. Ranges of peptide length of 4-50 residues, 7-50 residues, 4-25 residues 7-25, residues, 4-20 residues, 7-20 residues, and 3-15 residues, and 7-15 residues are contemplated. The number of consecutive MUC1 residues may be 3, 4, 5, 6, 7, 8, 9, 10, 11, 12, 13, 14, 15, 16, 17, 18, 19 or 20. Ranges of consecutive residues of 4-20 residues, 5-20 residues, 6-20 residues, 7-20 residues, 4-15 residues, 5-15 residues, 6-15 residues and 7-15 residues are contemplated.

The present invention may utilize L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

As mentioned above, the present invention contemplates fusing or conjugating a cell delivery domain (also called a cell delivery vector, or cell transduction domain). Such domains are well known in the art and are generally characterized as short amphipathic or cationic peptides and peptide derivatives, often containing multiple lysine and arginine resides (Fischer, 2007). Of particular interest are poly-D-Arg and poly-D-Lys sequences (e.g., dextrorotary residues, eight residues in length).

TABLE 1

| | SEQ |
|---|---|
| CDD/CTD PEPTIDES | ID NO |
| QAATATRGRSAASRPTERPRAPARSASRPRRPVE | 5 |
| RQIKIWFQNRRMKWKK | 6 |
| RRMKWKK | 7 |
| RRWRRWWRRWWRRWRR | 8 |
| RGGRLSYSRRRFSTSTGR | 9 |
| YGRKKRRQRRR | 10 |
| RKKRRQRRR | 11 |
| YARAAARQARA | 12 |
| RRRRRRRR | 13 |
| KKKKKKKK | 14 |
| GWTLNSAGYLLGKINLKALAALAKXIL | 15 |
| LLILLRRRIRKQANAHSK | 16 |
| SRRHHCRSKAKRSRHH | 17 |
| NRARRNRRRVR | 18 |
| RQLRIAGRRLRGRSR | 19 |
| KLIKGRTPIKFGK | 20 |
| RRIPNRRPRR | 21 |
| KLALKLALKALKAALKLA | 22 |
| KLAKLAKKLAKLAK | 23 |
| GALFLGFLGAAGSTNGAWSQPKKKRKV | 24 |
| KETWWETWWTEWSQPKKKRKV | 25 |
| GALFLGWLGAAGSTMGAKKKRKV | 26 |
| MGLGLHLLVLAAALQGAKSKRKV | 27 |
| AAVALLPAVLLALLAPAAANYKKPKL | 28 |
| MANLGYWLLALFVTMWTDVGLCKKRPKP | 29 |
| LGTYTQDFNKFHTFPQTAIGVGAP | 30 |
| DPKGDPKGVTVTVTVTVTGKGDPXPD | 31 |
| PPPPPPPPPPPPPP | 32 |
| VRLPPPVRLPPPVRLPPP | 33 |
| PRPLPPPRPG | 34 |
| SVRRRPRPPYLPRPRPPPFFPPRLPPRIPP | 35 |
| TRSSRAGLQFPVGRVHRLLRK | 36 |
| GIGKFLHSAKKFGKAFVGEIMNS | 37 |
| KWKLFKKIEKVGQNIRDGIIKAGPAVAVVGQATQIAK | 38 |
| ALWMTLLKKVLKAAAKAALNAVLVGANA | 39 |
| GIGAVLKVLTTGLPALISWIKRKRQQ | 40 |
| INLKALAALAKKIL | 41 |
| GFFALIPKIISSPLPKTLLSAVGSALGGSGGQE | 42 |
| LAKWALKQGFAKLKS | 43 |
| SMAQDIISTIGDLVKWIIQTVNXFTKK | 44 |
| LLGDFFRKSKEKIGKEFKRIVQRIKQRIKDFLANLVPRTES | 45 |
| LKKLLKKLLKKLLKKLLKKL | 46 |
| KLKLKLKLKLKLKLKLKL | 47 |
| PAWRKAFRWAWRMLKKAA | 48 |

Also as mentioned above, peptides modified for in vivo use by the addition, at the amino- and/or carboxyl-terminal ends, of a blocking agent to facilitate survival of the peptide in vivo are contemplated. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. These agents can be added either chemically during the synthesis of the peptide, or by recombinant DNA technology by methods familiar in the art. Alternatively, blocking agents such as pyroglutamic acid or other molecules known in the art can be attached to the amino and/or carboxyl terminal residues.

It will be advantageous to produce peptides using the solid-phase synthetic techniques (Merrifield, 1963). Other peptide synthesis techniques are well known to those of skill in the art (Bodanszky et al., 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Aside from the twenty standard amino acids can be used, there are a vast number of “non-standard” amino acids. Two of these can be specified by the genetic code, but are rather rare in proteins. Selenocysteine is incorporated into some proteins at a UGA codon, which is normally a stop codon. Pyrrolysine is used by some methanogenic archaea in enzymes that they use to produce methane. It is coded for with the codon UAG. Examples of non-standard amino acids that are not found in proteins include lanthionine, 2-aminoisobutyric acid, dehydroalanine and the neurotransmitter gamma-aminobutyric acid. Non-standard amino acids often occur as intermediates in the metabolic pathways for standard amino acids—for example ornithine and citrulline occur in the urea cycle, part of amino acid catabolism. Non-standard amino acids are usually formed through modifications to standard amino acids. For example, homocysteine is formed through the transsulfuration pathway or by the demethylation of methionine via the intermediate metabolite S-adenosyl methionine, while hydroxyproline is made by a posttranslational modification of proline.

In one aspect, the present invention focuses on peptides comprising the sequence CQCRRK (SEQ ID NO:4). Having identified this key structure in MUC1 oligomer formation, the inventor also contemplates that variants of the CQCRRK (SEQ ID NO:4) sequence may be employed. For example, certain non-natural amino acids that satisfy the structural constraints of the CQCRRK (SEQ ID NO:4) sequence may be substituted without a loss, and perhaps with an improvement in, biological function. In addition, the present inventor also contemplates that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Methods for generating specific structures have been disclosed in the art. For example, α-helix mimetics are disclosed in U.S. Pat. Nos. 5,446,128; 5,710,245; 5,840,833; and 5,859,184. Methods for generating conformationally restricted β-turns and β-bulges are described, for example, in U.S. Pat. Nos. 5,440,013; 5,618,914; and 5,670,155. Other types of mimetic turns include reverse and γ-turns. Reverse turn mimetics are disclosed in U.S. Pat. Nos. 5,475,085 and 5,929,237, and γ-turn mimetics are described in U.S. Pat. Nos. 5,672,681 and 5,674,976.

A particular modification is in the context of peptides as therapeutics is the so-called “Stapled Peptide” technology of Aileron Therapeutics. The general approach for “stapling” a peptide is that two key residues within the peptide are modified by attachment of linkers through the amino acid side chains. Once synthesized, the linkers are connected through a catalyst, thereby creating a bridge that physically constrains the peptide into its native α-helical shape. In addition to helping retain the native structure needed to interact with a target molecule, this conformation also provides stability against peptidases as well as promotes cell-permeating properties.

More particularly, the term “peptide stapling” may encompasses the joining of two double bond-containing sidechains, two triple bond-containing sidechains, or one double bond-containing and one triple bond-containing side chain, which may be present in a polypeptide chain, using any number of reaction conditions and/or catalysts to facilitate such a reaction, to provide a singly “stapled” polypeptide. In a specific embodiment, the introduction of a staple entails a modification of standard peptide synthesis, with α-methy, α-alkenyl amino acids being introduced at two positions along the peptide chain, separated by either three or six intervening residues (i+4 or i+7). These spacings place the stapling amino acids on the same fact of the α-helix, straddling either one (i+4) or two (i+7) helical turns. The fully elongated, resin-bound peptide can be exposed to a ruthenium catalyst that promotes cross-linking of the alkenyl chains through olefin metathesis, thereby forming an all-hydrocarbon macrocyclic cross-link. U.S. Pat. Nos. 7,192,713 and 7,183,059, and U.S. Patent Publications 2005/02506890 and 2006/0008848, describing this technology, are hereby incorporated by reference. See also Schafmeister et al. (2000); Walensky et al. (2004). Additionally, the term “peptide stitching” refers to multiple and tandem “stapling” events in a single peptide chain to provide a “stitched” (multiply stapled) polypeptide, each of which is incorporated herein by reference. See WO 2008/121767 for a specific example of stitched peptide technology.

7. Ligand Traps

The present invention contemplates the design, production and use of various MUC1 ligand traps. The contemplated ligand traps will have three elements: at least a portion of the MUC1-ED, a linker, and at least a portion of an immunoglobulin Fc sequence. Each of these elements is described in greater detail below.

In general, the peptides will be 50 residues or more in length comprising consecutive residues of MUC1-ED. The overall length may be 50, 60, 70, 80, 90, 100 or more residues. Ranges of peptide length of 50-60 residues, 50-70 residues, 50-80 residues 50-90, residues, 50-100 residues, 50-75 residues and 75-100 residues are contemplated. The number of consecutive MUC1 residues may be 10, 11, 12, 13, 14, 15, 16, 17, 18, 19, 20, 25, 30, 35, 40, 45, 50, 51, 52, 53, 54, 55, 56, 57, 58 or 59 residues. Ranges of consecutive residues of 10-20 residues, 15-20 residues, 15-25 residues, 10-30 residues, 10-40 residues, 10-50 residues, 10-59 residues and 20-59 residues are contemplated.

The ligand trap peptides may utilize L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

As mentioned above, peptides modified for in vivo use by the addition, at the amino- and/or carboxyl-terminal ends, of a blocking agent to facilitate survival of the peptide in vivo are contemplated. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. These agents can be added either chemically during the synthesis of the peptide, or by recombinant DNA technology by methods familiar in the art. Alternatively, blocking agents such as pyroglutamic acid or other molecules known in the art can be attached to the amino and/or carboxyl terminal residues.

The Fc (fragment, crystallizable) region of immunoglobulin interacts with the Fc receptor on certain cells. The constant region is identical in all antibodies of the same isotype, but differs in antibodies of different isotypes. There are five types of mammalian Ig heavy chains, denoted by the Greek letters: α, δ, ε, γ, and μ the constant regions of which dictate the structure of the Fc. Distinct heavy chains differ in size and composition; α and γ contain approximately 450 amino acids, while μ and ε have approximately 550 amino acids. Heavy chains γ, α and δ have a constant region composed of three tandem (in a line) Ig domains, and a hinge region for added flexibility; heavy chains μ and ε have a constant region composed of four immunoglobulin domains.

The MUC1-ED-Trap can be the 1-59 amino acids of MUC1-ED N-terminal to the plasma membrane domain fused with the constant region (Fc) of human or mouse IgG1. Additional MUC1-ED-Traps can be created where the constant region (Fc) of human or mouse IgG1 are fused with different portions of MUC1-ED (1-59 aa) and spaced with a linker sequences. Several other MUC1-ED-Traps can be used in which the highly positively charged amino acids from the MUC1-ED 1-59 domain can be excised. Moreover, a minor stretch of highly basic amino acids in MUC1-ED 1-59 can be deleted to generate a variant MUC1-ED-Trap for better PK characteristics.

Linkers or cross-linking agents may be used to fuse MUC1-ED segments to the constant region (Fc) of human or mouse IgG1 sequences. Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidino-, indole-, or carboxyl-specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described in U.S. Pat. No. 5,889,155, specifically incorporated herein by reference in its entirety. The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides. In instances where a particular peptide does not contain a residue amenable for a given cross-linking reagent in its native sequence, conservative genetic or synthetic amino acid changes in the primary sequence can be utilized.

The inventors constructed the Fc-MUC1-p59 chimeric protein connected by a (GGGGS)₃ linker. This chimeric protein can also be constructed by flexible linkers such as (GGGGS)ₙ where n=2-5. Moreover, helical linkers such as (EAAAK). where n=2-6 can also be used to provide proper conformation to the chimeric protein. The various sequences of the flexible linkers can be:

(SEQ ID NO: 49)
GGGGS GGGGS
(SEQ ID NO: 50)
GGGGS GGGGS GGGGS
(SEQ ID NO: 51)
GGGGS GGGGS GGGGS GGGGS
(SEQ ID NO: 52)
GGGGS GGGGS GGGGS GGGGS GGGGS

The various sequences of the helical linkers can be:

(SEQ ID NO: 53)
EAAAK EAAAK
(SEQ ID NO: 54)
EAAAK EAAAK EAAAK
(SEQ ID NO: 55)
EAAAK EAAAK EAAAK EAAAK

Other combinations are contemplated as well.

Alternatively or in addition to the linker described above, the ligand trap may include a glycosylation modification, in particular at what corresponds to residue Asn36 of the MUC1 58 reside ECD sequence. In the native molecule, this structural feature has been shown to be important in binding of MUC1 to molecules such as galactin-3, EGFR and ErbB2. This requirement with respect to galectin-3 has been demonstrated for the ligand trap as well.

U.S. Provisional Patent Application Ser. No. 61/524,978, filed Aug. 18, 2011, describing these ligand traps, is incorporated herein by reference.

8. MUC1 Dimerization Inhibitors

Flavones are a class of flavonoids based on the backbone of 2-phenylchromen-4-one (2-phenyl-1-benzopyran-4-one). Natural flavones include Apigenin (4′,5,7-trihydroxyflavone), Luteolin (3′,4′,5,7-tetrahydroxyflavone) and Tangeritin (4′,5,6,7,8-pentamethoxyflavone), chrysin (5,7-OH), 6-hydroxyflavone, baicalein (5,6,7-trihydroxyflavone), scutellarein (5,6,7,4′-tetrahydroxyflavone), wogonin (5,7-OH, 8-OCH₃). Synthetic flavones are Diosmin and Flavoxate.

Flavones are mainly found in cereals and herbs. In the West, the estimated daily intake of flavones is in the range 20-50 mg per day. In recent years, scientific and public interest in flavones has grown enormously due to their putative beneficial effects against atherosclerosis, osteoporosis, diabetes mellitus and certain cancers. Flavones intake in the form of dietary supplements and plant extracts has been steadily increasing. Flavones have effects on CYP (P450) activity which are enzymes that metabolize most drugs in the body.

Apigenin is a flavone that is the aglycone of several glycosides. It is a yellow crystalline solid that has been used to dye wool. Apigenin is a potent inhibitor of CYP2C9, an enzyme responsible for the metabolism of many pharmaceutical drugs in the body. Apigenin (4′,5,7-trihydroxyflavone) is commonly recognized as to mediated at least part of this chemopreventive action of vegetables and fruits in the cancerous process. Recently it was shown that Apigenin induces a process called autophagy (a kind of cellular dormancy) which may well explain it chemopreventive properties but at the same time induces resistance against chemotherapy.

Apigenin also has been shown to reverse the adverse effects of cyclosporine. Research has been conducted to study the effects of apigenin on reversal of cyclosporine A induced damage, and this was assessed by immunohistochemical estimation of expression of bcl-2, and estimation of apoptosis in histopathological sections. Cyclosporine A enhances the expression of transforming growth factor-β in the rat kidney, which signifies accelerated apoptosis. Therefore, transforming growth factor-β and apoptotic index may be used to assess apigenin and its effect on cyclosporine A induced renal damage.

PD98059. 2-(2′-amino-3′-methoxyphenyl)-oxanaphthalen-4-one, or PD98059, is a flavonoid and a potent inhibitor of mitogen-activated protein kinase kinase (MEK). Addition of PD98059 to rat liver cytosol just before the addition of TCDD suppressed TCDD binding and aryl hydrocarbon receptor (AHR) transformation, as measured by sucrose gradient centrifugation and electrophoretic mobility shift assays. These results suggest that PD98059 is a ligand for the AHR and functions as an AHR antagonist at concentrations commonly used to inhibit MEK and signaling processes that entail MEK activation.

Kaempferol. Kaempferol is a natural flavonoid that has been isolated from tea, broccoli, Delphinium, Witch-hazel, grapefruit, brussel sprouts, apples and other plant sources. Kaempferol is a yellow crystalline solid with a melting point of 276-278° C. It is slightly soluble in water but soluble in hot ethanol and diethyl ether. Many glycosides of kaempferol, such as kaempferitrin and astragalin, have been isolated as natural products from plants. Kaempferol consumption in tea and broccoli has been associated with reduced risk of heart disease and has antidepressant properties. An 8-year study found that three flavonols (kaempferol, quercetin, and myricetin) reduced the risk of pancreatic cancer by 23%.

Fisetin. Fisetin, an analogue of quercetin, is a brown pigment found in woody plants. It has antioxidant properties which protect cells against oxygen radical damage. It is also reported to inhibit xanthine oxidase, a free-radical generating enzyme and show and inhibit the oxidation of LDL (low density lipoprotein) by free radicals.

Morin. Morin (3,5,7,2′,4′-pentahydroxyflavone) is a flavonoid yellow color substance that can be isolated from Maclura pomifera (Osage orange), Maclura tinctoria (old fustic) and from leaves of Psidium guajava (common guava). It is an important bioactive compound interacting with nucleic acids, enzymes and protein. Oral administration offers protection against hyperammonemia by means of reducing blood ammonia, oxidative stress and enhancing antioxidant status in ammonium chloride-induced hyperammonemic rats. Enhanced blood ammonia, plasma urea, lipid peroxidation in circulation and tissues (liver and brain) of ammonium chloride-treated rats was accompanied by a significant decrease in the tissues levels of superoxide dismutase (SOD), catalase, reduced glutathione (GSH) and glutathione peroxidase (GPx). Morin administered to rats showed a significant reduction in ammonia, urea, lipid peroxidation with a simultaneous elevation in antioxidant levels.

Other Flavones. The general structure below provides additional/similar flavone structures for use in accordance with the present invention:

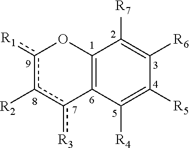

wherein

R₁ is H, —OH, ═O, substituted or unsubstituted alkyl(C₁₋₈), alkoxy(C₁₋₈), haloalkyl(C₁₋₈), substituted phenyl or unsubstituted phenyl, wherein if R₁ is ═O, Cγ-C₈ is a double bond;

R₂ is H, —OH, alkyl(C₁₋₈), substituted phenyl, unsubstituted phenyl, phenyl, phenyl thiazole, imidazole, pyrazole or furan;

R₃ is H, —OH, ═O, halogen, haloalkyl(C₁₋₈), substituted or unsubstituted alkyl(C₁₋₈), substituted phenyl or unsubstituted phenyl, wherein if R₃ is ═O, C₈-C₉ is a double bond;

R₄ is H or —OH;

R₅ is H, —OH, substituted or unsubstituted alkyl(C₁₋₈) or alkoxy(C₁₋₈), or OR₈, wherein R₈ is alkyl(C₁₋₈), an ester or an amide;

R₆ is H, —OH, substituted or unsubstituted alkyl(C₁₋₈) or alkoxy(C₁₋₈), or OR₈, wherein R₈ is alkyl(C₁₋₈), an ester or an amide; and

R₇ is H, —OH, or substituted or unsubstituted alkyl(C₁₋₈),

with the proviso that R₁ and R₃ cannot both be ═O.

When used in the context of a chemical group, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylimino); “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH₃ (see below for definitions of group(s) containing the term silyl, e.g., alkylsilyl).

The symbol “—” means a single bond, “═” means a double bond, and “≡” means triple bond. The symbol “ [symbol] ” represents a single bond or a double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation is unknown (e.g., either R or 5), the geometry is unknown (e.g., either E or Z) or the compound is present as mixture of conformation or geometries (e.g., a 50%/50% mixture).

When a group “R” is depicted as a “floating group” on a ring system, for example, in the formula:

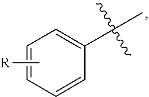

then R may replace any hydrogen atom attached to any of the ring atoms, including a depicted, implied, or expressly defined hydrogen, so long as a stable structure is formed.

When a group “R” is depicted as a “floating group” on a fused ring system, as for example in the formula:

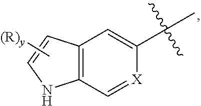

then R may replace any hydrogen attached to any of the ring atoms of either of the fuzed rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

When y is 2 and “(R)_{y}” is depicted as a floating group on a ring system having one or more ring atoms having two replaceable hydrogens, e.g., a saturated ring carbon, as for example in the formula:

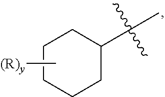

then each of the two R groups can reside on the same or a different ring atom. For example, when R is methyl and both R groups are attached to the same ring atom, a geminal dimethyl group results. Where specifically provided for, two R groups may be taken together to form a divalent group, such as one of the divalent groups further defined below. When such a divalent group is attached to the same ring atom, a spirocyclic ring structure will result.

When the point of attachment is depicted as “floating”, for example, in the formula:

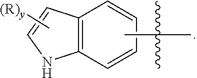

then the point of attachment may replace any replaceable hydrogen atom on any of the ring atoms of either of the fuzed rings unless specified otherwise.

In the case of a double-bonded R group (e.g., oxo, imino, thio, alkylidene, etc.), any pair of implicit or explicit hydrogen atoms attached to one ring atom can be replaced by the R group. This concept is exemplified below:

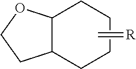

represents

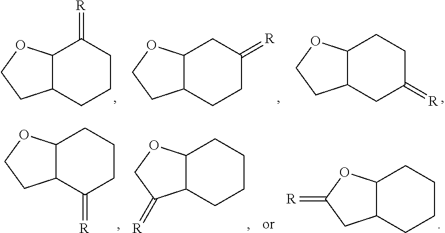

For the groups below, the following parenthetical subscripts further define the groups as follows: “(Cn)” defines the exact number (n) of carbon atoms in the group. “(Cn)” defines the maximum number (n) of carbon atoms that can be in the group, with the minimum number of carbon atoms in such at least one, but otherwise as small as possible for the group in question, e.g., it is understood that the minimum number of carbon atoms in the group “alkenyl_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and 1,4-cycloalkanediyl, are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and 1,4-cycloalkenediyl, are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), C₆H₄CH(CH₃)₂, C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: C₆H₄F, C₆H₄Cl, C₆H₄Br, C₆H₄I, —C₆H₄OH, —C₆H₄OCH₃, C₆H₄OCH₂CH₃, —C₆H₄OC(O)CH₃, C₆H₄NH₂, C₆H₄NHCH₃, C₆H₄N(CH₃)₂, C₆H₄CH₂OH, C₆H₄CH₂OC(O)CH₃, C₆H₄CH₂NH₂, —C₆H₄CF₃, C₆H₄CN, C₆H₄CHO, C₆H₄CHO, C₆H₄C(O)CH₃, C₆H₄C(O)C₆H₅, C₆H₄CO₂H, —C₆H₄CO₂CH₃, C₆H₄CONH₂, C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

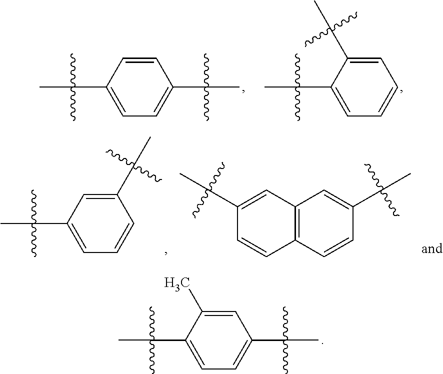

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are carbon, and wherein the divalent group further has at least one atom independently selected from the group consisting of N, O, F, Cl, Br, I, Si, P, and S.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two σ-bonds, with an aromatic carbon atom or nitrogen atom as the point of attachment, said carbon atom or nitrogen atom forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. Non-limiting examples of heteroarenediyl groups include:

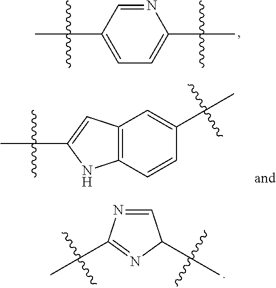

The term “substituted heteroarenediyl” refers to a divalent group, wherein the heteroarenediyl group is attached with two σ-bonds, with an aromatic carbon atom or nitrogen atom as points of attachment, said carbon atom or nitrogen atom forming part of one or more six-membered aromatic ring structure(s), wherein at least one of the ring atoms is nitrogen, oxygen or sulfur, and wherein the divalent group further has at least one atom independently selected from the group consisting of non-aromatic nitrogen, non-aromatic oxygen, non aromatic sulfur F, Cl, Br, I, Si, and P.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, C(O)CH(CH₂)₂, C(O)C₆H₅, C(O)C₆H₄CH₃, C(O)C₆H₄CH₂CH₃, COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group ═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

The term “fluoroalkyl” when used without the “substituted” modifier refers to an alkyl, as that term is defined above, in which one or more fluorines have been substituted for hydrogens. The groups, —CH₂F, —CF₂H, —CF₃, and —CH₂CF₃ are non-limiting examples of fluoroalkyl groups. The term “substituted fluoroalkyl” refers to a non-aromatic monovalent group with a saturated carbon atom as the point of attachment, a linear or branched, cyclo, cyclic or acyclic structure, at least one fluorine atom, no carbon-carbon double or triple bonds, and at least one atom independently selected from the group consisting of N, O, Cl, Br, I, Si, P, and S. The following group is a non-limiting example of a substituted fluoroalkyl: —CFHOH.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “substituted alkylphosphate” refers to the group —OP(O)(OH)(OR), in which R is a substituted alkyl, as that term is defined above.

The term “dialkylphosphate” when used without the “substituted” modifier refers to the group —OP(O)(OR)(OR′), in which R and R′ can be the same or different alkyl groups, or R and R′ can be taken together to represent an alkanediyl having two or more saturated carbon atoms, at least two of which are attached via the oxygen atoms to the phosphorus atom. Non-limiting examples of dialkylphosphate groups include: —OP(O)(OMe)₂, —OP(O)(OEt)(OMe) and —OP(O)(OEt)₂. The term “substituted dialkylphosphate” refers to the group —OP(O)(OR)(OR), in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached via the oxygen atoms to the phosphorous.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, C(S)CH(CH₂)₂, C(S)C₆H₅, C(S)C₆H₄CH₃, C(S)C₆H₄CH₂CH₃, C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, C(S)OCH₃, —C(S)OCH₂CH₃, C(S)OCH₂CH₂CH₃, C(S)OC₆H₅, C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: S(O)₂CH₃, S(O)₂CH₂CH₃, S(O)₂CH₂CH₂CH₃, —S(O)₂CH(CH₃)₂, —S(O)₂CH(CH₂)₂, —S(O)₂-cyclopentyl, and —S(O)₂-cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylsulfinyl” when used without the “substituted” modifier refers to the group —S(O)R, in which R is an alkyl, as that term is defined above. Non-limiting examples of alkylsulfinyl groups include: —S(O)CH₃, —S(O)CH₂CH₃, —S(O)CH₂CH₂CH₃, —S(O)CH(CH₃)₂, —S(O)CH(CH₂)₂, —S(O)-cyclopentyl, and —S(O)-cyclohexyl. The term “substituted alkylsulfinyl” refers to the group —S(O)R, in which R is a substituted alkyl, as that term is defined above. For example, —S(O)CH₂CF₃ is a substituted alkylsulfinyl group.

Similarly, the terms “alkenylsulfinyl”, “alkynylsulfinyl”, “arylsulfinyl”, “aralkylsulfinyl”, “heteroarylsulfinyl”, and “heteroaralkylsulfinyl” when used without the “substituted” modifier, refers to groups, defined as —S(O)R, in which R is alkenyl, alkynyl, aryl, aralkyl, heteroaryl, and heteroaralkyl, respectively, as those terms are defined above. When any of the terms alkenylsulfinyl, alkynylsulfinyl, arylsulfinyl, aralkylsulfinyl, heteroarylsulfinyl, and heteroaralkylsulfinyl is modified by “substituted,” it refers to the group —S(O)R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or —NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)⁺, —NH₂(CH₂CH₂CH₃)⁺, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or —NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′⁺, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, —SH(cyclopentyl)⁺, and —SH(cyclohexyl)⁺. The term “substituted alkylsulfonium” refers to the group —SRR′⁺, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺ is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers to —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A compound having a formula that is represented with a dashed bond is intended to include the formulae optionally having zero, one or more double bonds. Thus, for example, the structure

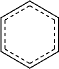

includes the structures

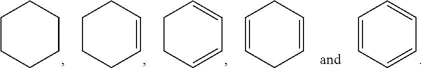

As will be understood by a person of skill in the art, no one such ring atom forms part of more than one double bond.

Any undefined valency on an atom of a structure shown in this application implicitly represents a hydrogen atom bonded to the atom.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002),

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerisation, that is, the number of repeat units linked together. When the value for “n” is left undefined, it simply designates repetition of the formula within the brackets as well as the polymeric nature of the material. The concept of a repeat unit applies equally to where the connectivity between the repeat units extends three dimensionally, such as in metal organic frameworks, cross-linked polymers, thermosetting polymers, etc.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five, and optionally amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

U.S. Ser. No. 13/045,033, filed Mar. 10, 2011, describing these compounds, is incorporated herein by reference.

9. Dosage

The skilled artisan is directed to “Remington's Pharmaceutical Sciences” 15th Ed., in particular pages 33:624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancers.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Culture.

Human MCF-10A mammary epithelial cells were grown in mammary epithelial growth medium (MEGM, Lonza). Human MCF-7 and MDA-MB-468 breast cancer cells were cultured in Dulbecco's modified Eagle's medium with 10% heat-inactivated fetal bovine serum (FBS), 100 U/ml penicillin, 100 μg/ml streptomycin and 2 mM L-glutamine. Human BT-549 cells were maintained in RPMI-1640 medium (ATCC) with 10% FBS and 0.023 IU/ml insulin. Cells were treated with EGF (Sigma), heregulin beta 1 (HRG; Neomarkers), cycloheximide (CHX, Calbiochem), U0126 (Calbiochem), LY294002 (Caymam Chemical Company), BEZ-235 (Selleckchem) and rapamycin Cell Signaling Technology). In certain experiments, MCF-10A cells were serum-starved overnight, and inhibitors were added 2.5 h prior to EGF or HRG treatment.

eIF4A Inhibitors.

Silvestrol (36), CR-1-31-B (30), and its enantiomer CR-1-30-B (30) were synthesized according to literature procedures.

Immunoblot Analysis.

Lysates from subconfluent cells were immunoblotted with anti-MUC1-C(AbS; Neomarkers), anti-β-actin (Sigma), anti-p-AKT, anti-AKT, anti-p-ERK1/2, anti-ERK1/2, anti-p-S6K1, anti-S6K1, anti-PCDC4, anti-eIF4E, eIF4A (Cell Signaling Technology) and anti-EGFR (Santa Cruz Technology) Immune complexes were detected with horseradish peroxidase-conjugated secondary antibodies and enhanced chemiluminescence (Amersham Biosciences). Intensity of certain signals was determined by densitometric scanning.

Luciferase Assays.

Control pGL3 or pGL3-MUC1-promoter constructs were transfected with the Renilla plasmid into cells in the presence of Lipofectamine. At 24 h after transfection, cells were serum-starved overnight, and then treated with EGF (100 ng/ml) for 5 h. Luciferase reporter activity was measured using the Promega Dual Glo kit.

Real-Time PCR.

Total RNA was isolated from cells using an RNeasy Mini kit (Qiagen). cDNAs were synthesized with 0.3-1 μg RNA using the first-strand cDNA synthesis kit (Invitrogen). The SYBR green qPCR assay kit (Applied Biosystems) was used with 5 μl of 20-fold diluted cDNA from each sample, and the samples were amplified with the ABI Prism 7300 machine (Applied Biosystems).

Coimmunoprecipitation Experiments.

MCF-10A cells were treated with EGF and lysed for 30 min on ice. Cell lysates were incubated with control IgG or anti-EGFR overnight at 4° C. with agitation. Protein G-Sepharose beads (GE Health Care Life Sciences) were added and the cell lysates were incubated for another 2 h Immune complexes were collected, washed in lysis buffer and subjected to immunoblotting with anti-MUC1-C and anti-EGFR.

Confocal Microscopy.

MCF-10A cells were seeded onto a 6-well plate with sterile cover slides. After overnight serum starvation, cells were treated with EGF for 24 h. Cells were fixed in 100% acetone, blocked with 5% milk in PBS and stained with an anti-EGFR monoclonal antibody (Santa Cruz Biotechnology), anti-MUC1-C(Neomarkers) and Hoechst 33258 (Invitrogen). The cover slides were mounted onto microscope slides using Prolong Gold antifade reagent (Invitrogen) and imaged by confocal microscopy.

Assessment of Cell Cycle Distribution.

Cells were fixed in ice-cold 100% ethanol overnight, washed with PBS, incubated with 100 μg/ml RNase for 30 min at 37° C., stained with 10 μg/ml propidium iodide at room temperature for 30 min, and analyzed by flow cytometry.

### Example 2

### Results

Growth Factor Stimulation Induces MUC1-C Expression.

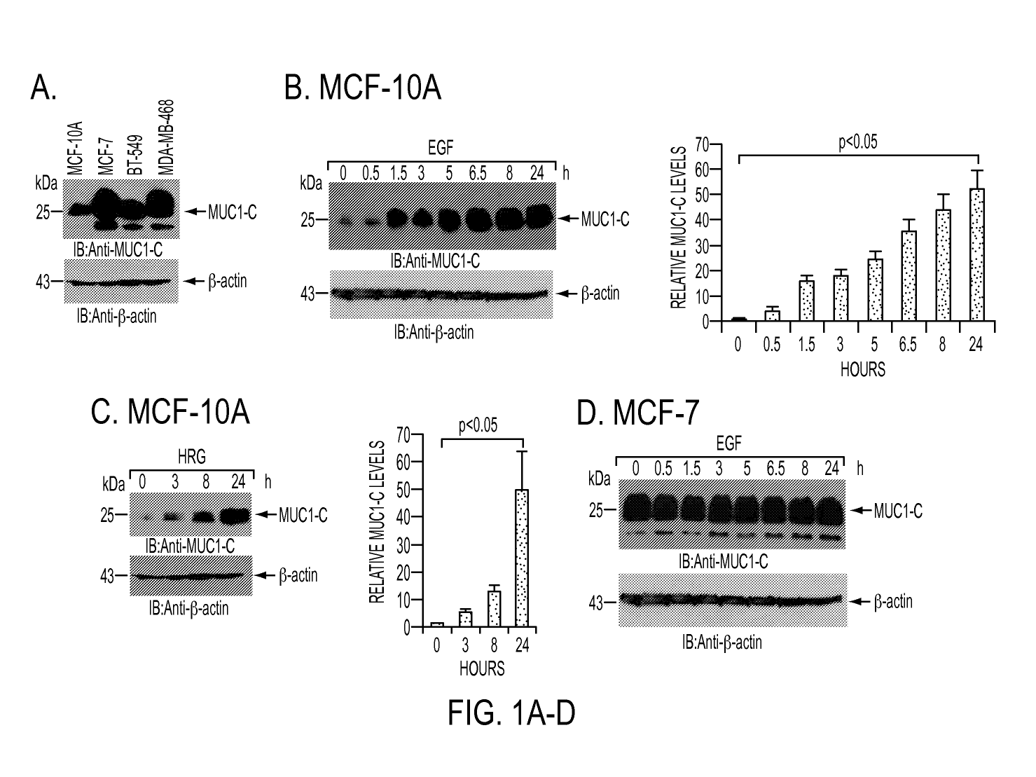

Abundance of the ˜25 kDa MUC1-C protein is relatively lower in non-malignant MCF-10A breast epithelial cells as compared to that MCF-7, BT-549 and MDA-MB-468 breast cancer cells (FIG. 1A). Consequently, the inventor reasoned that MCF-10A cells might represent a potential model to study mechanisms responsible for the overexpression of MUC1-C in breast cancer cells. In this context, the inventor found that stimulation of MCF-10A cells with EGF is associated with marked upregulation of MUC1-C expression with an increase of 65-fold at 24 h compared to baseline levels (FIG. 1B, left). Densitometric scanning of the signals from repetitive experiments further demonstrated a time-dependent increase in MUC1-C abundance (FIG. 1B, right). Treatment of MCF-10A cells with heregulin (HRG) was similarly associated with a substantial increase in MUC1-C abundance (58-fold at 24 h compared to baseline) (FIG. 1C, left and right). By contrast, EGF had no apparent effect on MUC1-C levels in MCF-7 cells (FIG. 1D). Stimulation of MCF-7 cells with HRG also had no effect on MUC1-C abundance (data not shown), indicating that MUC1-C expression is inducible by growth factors in MCF-10A, but not MCF-7, cells.

MUC1-C Translation is Induced by the PI3K->AKT->mTOR Pathway.

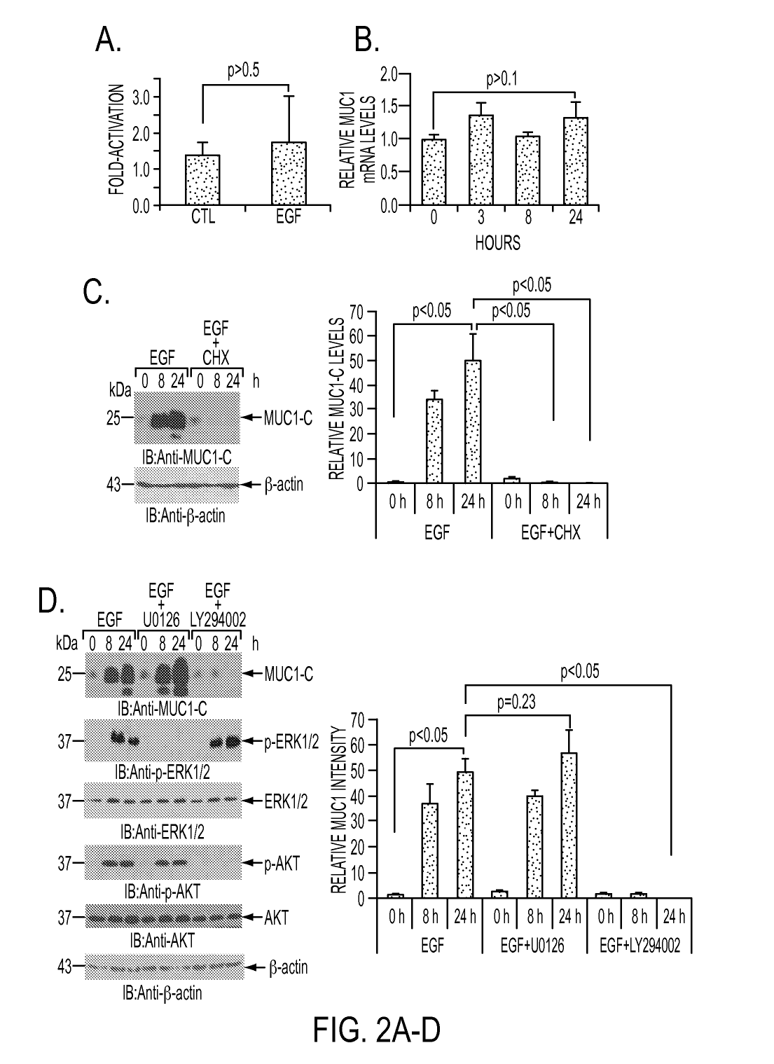

To define the basis for growth factor-induced increases in MUC1-C expression, the inventors asked if the upregulation in levels is mediated by transcriptional and/or post-transcriptional mechanisms. EGF stimulation of MCF-10A cells had no significant effect on activation of the MUC1 gene promoter in MCF-10A cells (FIG. 2A). Moreover, EGF had no apparent effect on MUC1 mRNA levels as determined by qRT-PCR, indicating that the increase in MUC1-C protein is regulated at the post-transcriptional level (FIG. 2B). As a control, inhibition of protein synthesis with cycloheximide (CHX) blocked EGF-induced increases in MUC1-C abundance (FIG. 2C, left and right), confirming that translation of MUC1-C is upregulated in the response to growth factor stimulation. Of note, the basal levels of MUC1-C in MCF-10A cells varies among experiments as a result of differences in exposure times used for detection of the signals. Certain signaling pathways, such as MEK->ERK1/2 and PI3K->AKT, have been linked to the activation of protein translation (Sonenberg et al., 2009). To assess potential involvement of ERK1/2 and/or PI3K in the regulation of MUC1-C translation, EGF-stimulated MCF-10A cells were treated with the dual ERK1/2 inhibitor, U0126 (27), or the PI3K inhibitor, LY294002 (Workman et al., 2010). Inhibition of PI3K, but not ERK1/2, blocked EGF-mediated induction of MUC1-C expression (FIG. 2D, left and right).

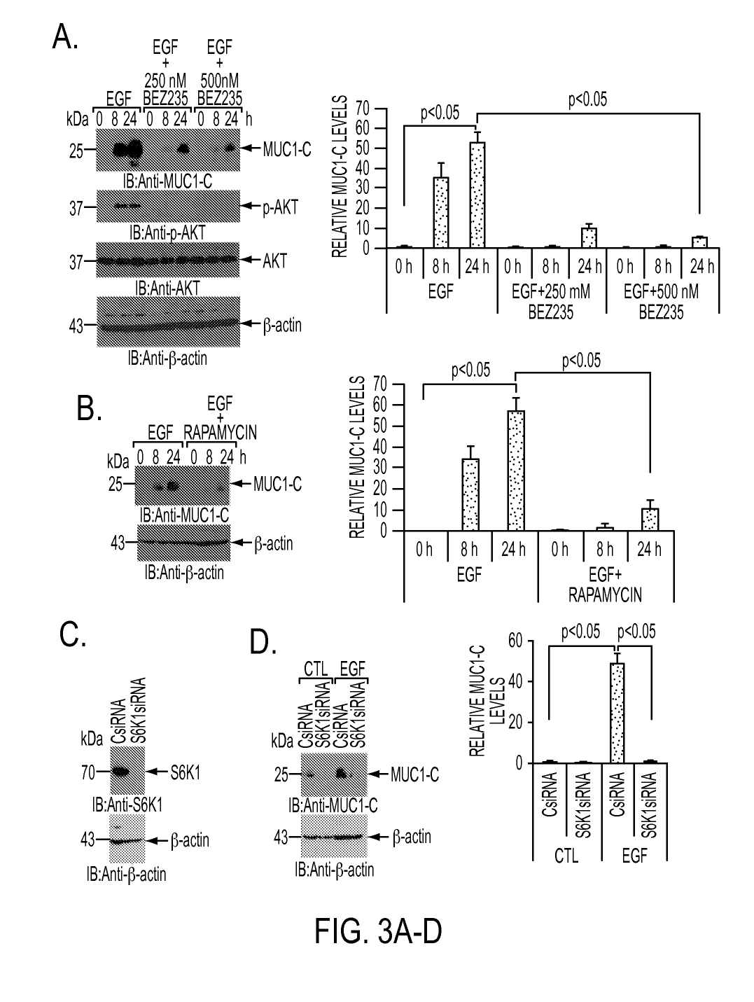

To extend this analysis, experiments were performed with BEZ235, an inhibitor of PI3K and mTOR (Kong et al., 2010). As found with LY294002, BEZ235 blocked EGF-induced increases in MUC1-C abundance (FIGS. 3A, left and right). In concert with these results, treatment of EGF-stimulated MCF-10A cells with rapamycin, an allosteric inhibitor of mTOR (Sonenberg et al., 2009), was also associated with a block in the induction of MUC1-C expression (FIG. 3B, left and right). mTOR is part of the mTORC1 complex, which phosphorylates the ribosomal protein S6 kinase 1 (S6K1) and thereby contributes to the initiation of translation (Sonenberg et al., 2009). The demonstration that silencing S6K1 (FIG. 3C) inhibits EGF-induced upregulation of MUC1-C levels (FIG. 3D, left and right) provided further support for involvement of the PI3K->AKT->mTORC1->S6K1 pathway in the activation of MUC1-C translation.

Inhibiting Cap-Dependent Translation Blocks Growth Factor-Induced Increases in MUC1-C Abundance.

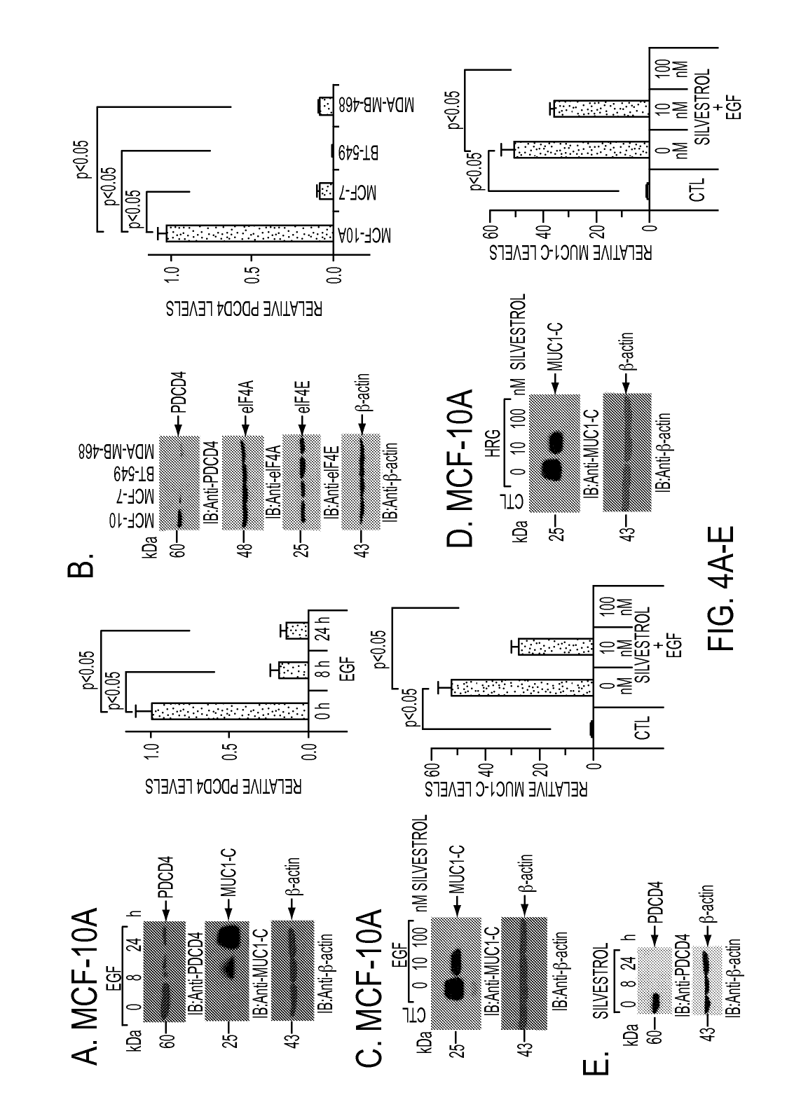

S6K1-mediated activation of the eIF4A RNA helicase is essential for unwinding of certain 5′UTRs and induction of translation (Ma and Blenis, 2009). To address the potential role of eIF4A, the inventor first examined expression of the tumor suppressor programmed cell death protein 4 (PDCD4), which inhibits the eIF4A RNA helicase activity (Palamarchuk et al., 2005; Jansen et al., 2005). Stimulation of MCF-10A cells with EGF was associated with downregulation of PDCD4 levels in association with increases in MUC1-C abundance (FIG. 4A, left and right). Moreover and in concert with the constitutive upregulation of MUC1-C, PDCD4 was low to undetectable in MCF-7, BT-549 and MDA-MB-468 cells (FIG. 4B, left and right). In contrast to PDCD4, there was little difference among these cells in terms of eIF4A and eIF4E expression (FIG. 4B, left). Whereas PDCD4 inhibits the eIF4A RNA helicase, EGF-stimulated MCF-10A cells were treated with silvestrol, an inhibitor of eIF4A RNA helicase activity (Bordeleau et al., 2008). In this context, there is presently no assay for monitoring eIF4A activity in cells and silvestrol is used to assess dependence on this helicase (Lucas et al., 2009; Schatz et al., 2011). Notably, silvestrol blocked EGF-mediated activation of MUC1-C translation Notably, silvestrol blocked EGF-mediated activation of MUC1-C translation in a dose-dependent manner (FIG. 4C, left and right). Similar dose-dependent inhibitory effects were obtained when HRG-stimulated MCF-10A cells were treated with silvestrol (FIG. 4D, left and right). MCF-10A cells were also treated with silvestrol and monitored for effects on PDCD4 expression. The results demonstrate that silvestrol treatment is associated with a marked decrease in PDCD4 abundance (FIG. 4E). These findings indicate that (i) growth factor-induced MUC1 translation is associated with degradation of PDCD4 and activation of the eIF4A RNA helicasehelicase, and (ii) silvestrol blocks growth-factor-induced MUC1 translation by inhibiting eIF4A activity. Silvestrol also decreased PDCD4 expression in a potential feedback response to the inhibition of eIF4A activity.

Upregulation of MUC1-C Expression Contributes to EGFR-Mediated Signaling.

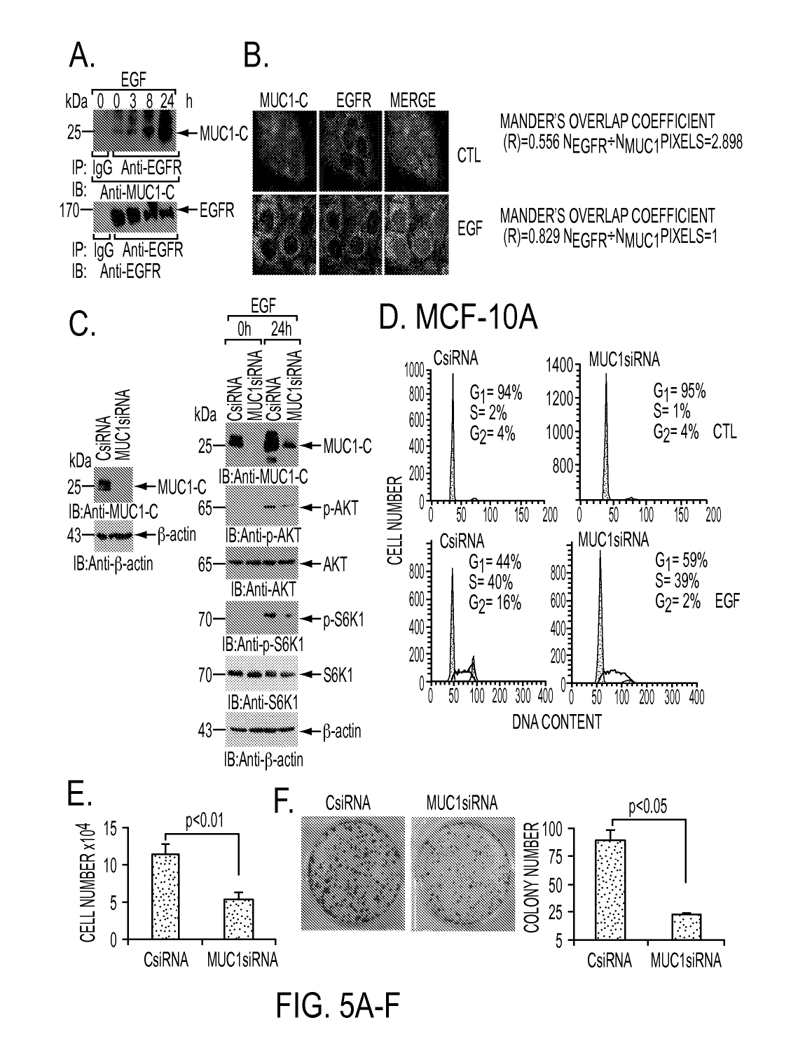

MUC1-C forms complexes with EGFR at the cell membrane of breast cancer cells (Ramasamy et al., 2007). Coimmunoprecipitation studies were therefore performed to determine whether the upregulation of MUC1-C expression affects the formation of EGFR/MUC1-C complexes. Analysis of anti-EGFR precipitates demonstrated a time-dependent increase in the association of EGFR and MUC1-C(FIG. 5A). In addition, the increases in EGFR-MUC1-C complexes were associated with partial downregulation of EGFR levels observed in the response to EGF stimulation (FIG. 5A). These results were extended with confocal microscopy studies of EGF-stimulated MCF-10A cells demonstrating colocalization of EGFR and MUC1-C at the cell surface (FIG. 5B, left). Analysis of the images using Image J (Li et al., 2004) confirmed a significant increase in EGFR and MUC1-C colocalization after EGF stimulation as supported by an enhanced Mander's overlap coefficient with a N_{EGFR}/N_{MUC1-C} 238 pixels=1 (FIG. 5B, right). To further assess the functional role of MUC1-C, the inventor generated MCF-10A cells that were stably silenced for MUC1-C expression (FIG. 5C, left). Whereas MUC1-C contributes to EGFR-mediated activation of the PI3K->AKT pathway (Raina et al., 2004), studies were performed to assess the effects of MUC1-C silencing on EGF-induced PI3K->AKT signaling (FIG. 4C, right). Notably, EGF-induced increases in p-AKT and p-S6K1 were suppressed in association with the silencing of MUC1-C(FIG. 5C, right), demonstrating that MUC1-C contributes to EGFR-mediated signaling. In concert with these results, EGF-induced cell cycle progression was attenuated as a result of MUC1-C silencing with increases in G1 phase and decreases in G2 phase (FIG. 5D). In addition, decreases in MUC1-C abundance attenuated EGF-stimulated MCF-10A cell growth (FIG. 5E) and colony formation (FIG. 5F, left and right). These findings indicate that EGF stimulates MUC1-C expression and, in turn, MUC1-C promotes EGFR-induced PI3K->AKT signaling and cell growth.

eIF4A RNA Helicase Activity Confers Translation of MUC1-C in Growth Factor-Stimulated MCF-10A Cells.

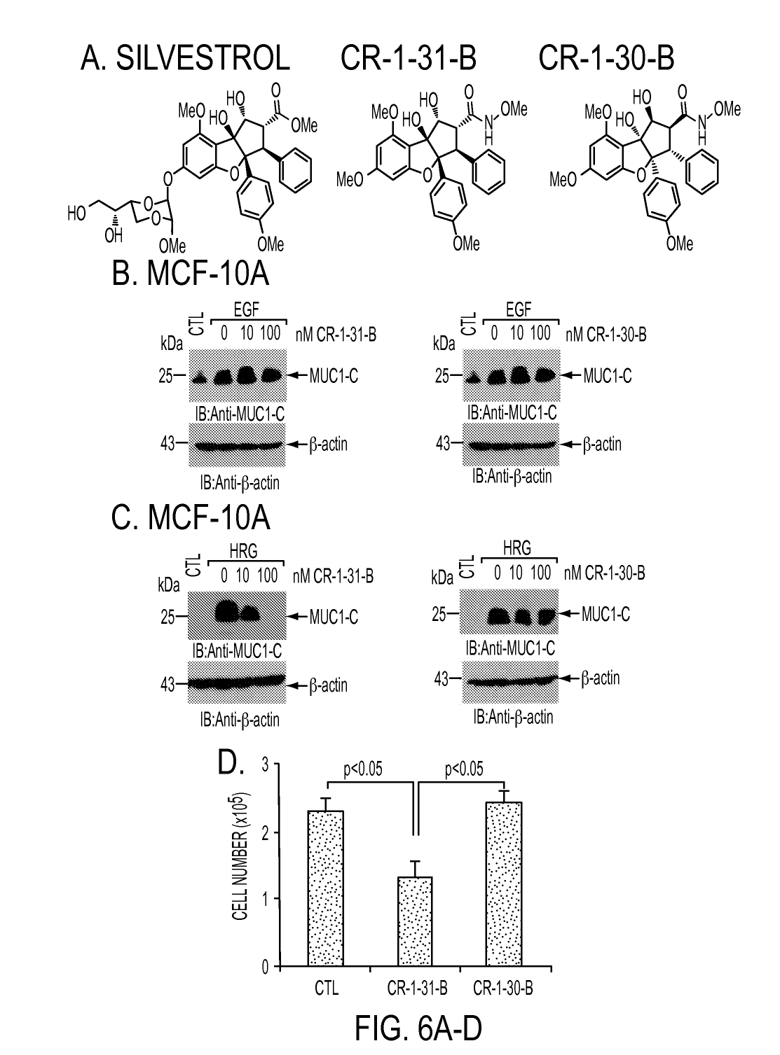

To confirm the notion that eIF4A RNA helicase activity induces MUC1-C translation, the inventors assessed the effects of a silvestrol analog, designated CR-1-31-B, and its enantiomer CR-1-259 30-B, which is inactive against eIF4A (Rodrigo et al. 2012) (FIG. 6A). As found with silvestrol, CR-1-31-B treatment of EGF-stimulated MCF-10A cells was associated with a dose-dependent decrease in MUC1-C abundance (FIG. 6B, left). By contrast, the inactive CR-1-30-B had no effect on EGF-induced MUC1-C expression (FIG. 6B, right). Treatment of MCF-10A cells with CR-1-31-B, but not the inactive CR-1-30-B, also blocked HRG-induced increases in MUC1-C abundance (FIG. 6C, left and right). In concert with the effects of inhibiting the eIF4A RNA helicase on the cap-dependent translation of multiple oncoproteins, growth of MCF-10A cells in response to EGF was attenuated by CR-1-31-B and not CR-1-30-B (FIG. 6D).

PI3K->AKT Pathway Contributes to MUC1-C Translation in Breast Cancer Cells.

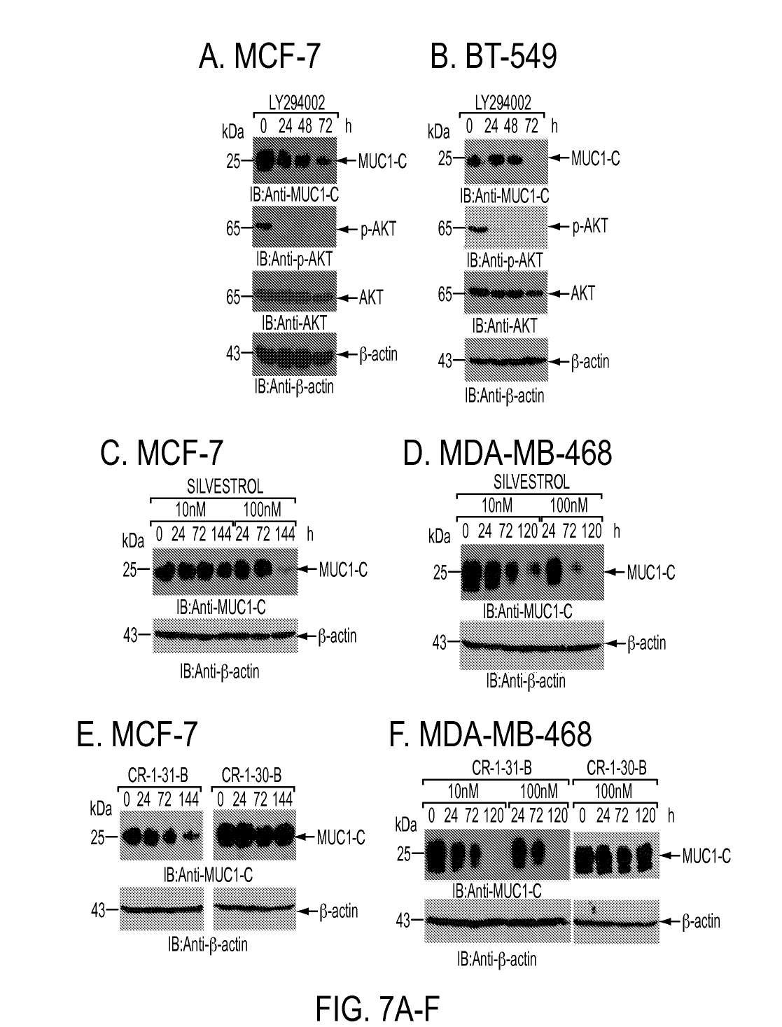

Based on the results obtained in MCF-10A cells, the inventor asked if PI3K->AKT-induced activation of MUC1-C translation contributes to the constitutive overexpression of MUC1-C in breast cancer cells. Accordingly, treatment of MCF-7 cells with LY294002 was associated with progressive decreases in MUC1-C abundance that corresponded with inhibition of p-AKT (FIG. 7A). In addition, downregulation of MUC1-C protein levels by LY294002 occurred in the absence of a detectable effect on MUC1 mRNA levels (data not shown). Similar results were obtained with LY294002-treated BT-549 breast cancer cells (FIG. 7B), indicating that PI3K->AKT signaling contributes to MUC1-C overexpression. To extend these observations to the regulation of MUC1-C translation, the inventor treated breast cancer cells with silvestrol. Exposure of MCF-7 cells to 10 nM silvestrol had a limited effect on MUC1-C levels (FIG. 7C). Moreover, treatment with 100 nM silvestrol was associated with a more pronounced decrease in MUC1-C abundance (FIG. 7C). Treatment of BT-549 cells resulted in a similar dose-dependent effect of silvestrol on MUC1-C levels (data not shown). Treatment of MDA-MB-468 breast cancer cells also demonstrated decreases in MUC1-C that were clearly detectable in response to 10 and 100 nM silvestrol (FIG. 7D). Treatment of MCF-7 cells with 100 nM CR-1-31-B was associated with downregulation of MUC1-C abundance (FIG. 7E, left). By contrast, the inactive CR-1-30-B enantiomer had no apparent effect (FIG. 7E, right). The inventors also found that 100 nM CR-1-31-B, but not the inactive CR-1-30-B, decreases MUC1-C expression in BT-549 cells (data not shown). In addition, treatment of MDA-MB-468 breast cancer cells with 10 and 100 nM CR-1-31-B was associated with decreases in MUC1-C abundance (FIG. 7F). These findings collectively indicated that PI3K->AKT signaling activates eIF4A RNA helicase-mediated translation of MUC1-C in breast cancer cells.

### Example 3

### Discussion

Growth Factor-Induced PI3K->AKT Signaling Induces MUC1-C Translation.

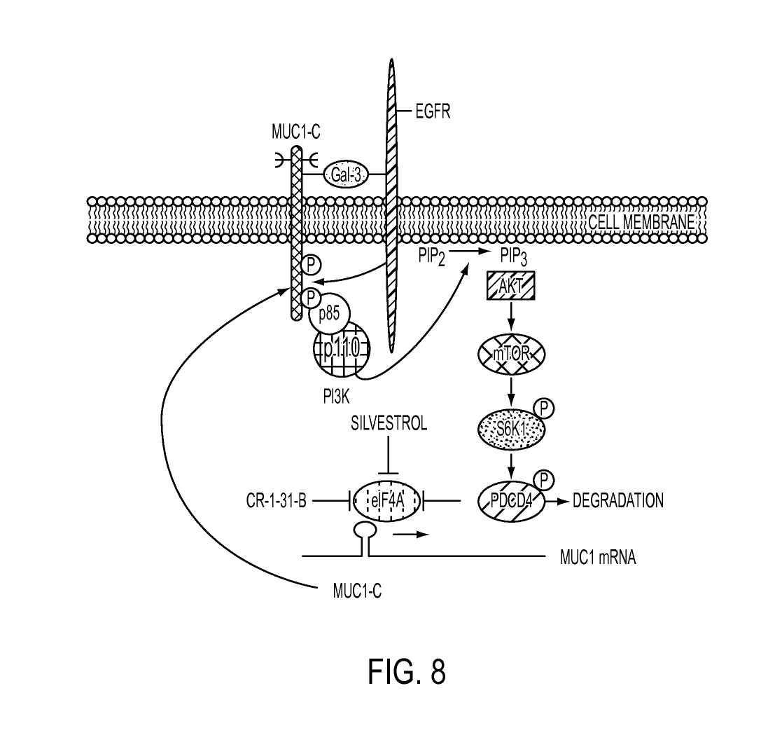

The MUC1 heterodimer is localized at the apical border of normal epithelial cells and is thus sequestered from EGFR and other RTKs that reside at the basal-lateral borders (Kufe, 2009). In the response to stress, epithelial cells lose apical-basal polarity in association with activation of a proliferation and survival program (Vermeer et al., 2003). Under these circumstances, the MUC1-C subunit is now repositioned to form complexes with RTKs, such as EGRF, and promote their activation of downstream growth and survival signals (FIG. 8) (Kufe, 2009). The present studies demonstrate that stimulation of non-malignant MCF-10A mammary epithelial cells with EGF results in pronounced increases in MUC1-C translation. Similar effects were observed with HRG, an activator of ErbB2, indicating that this increase in MUC1-C levels is not restricted to EGFR stimulation. Indeed, our results do not exclude the possibility that activation of non-ErbB RTKs also induce MUC1-C expression. The inventor also found that EGF- and HRG-induced increases in MUC1-C abundance were suppressed by inhibitors of the PI3K->AKT pathway and not those that block MEK->ERK1/2 signaling (FIG. 8). AKT controls protein synthesis at multiple levels, including ribosome biogenesis, translation initiation and elongation, leading to changes in translation of select mRNAs (Hsieh et al., 2011). The present results demonstrate that like other oncoproteins, for example cyclin D1, MYC and MCL1 (Ma and Blenis, 2009; Sonenberg and Hinnebusch, 2009), translation of MUC1-C is selectively induced by growth factor stimulation and activation of AKT signaling. The functional significance of upregulating MUC1-C translation is supported by the findings that MUC1-C in turn forms complexes with EGFR and promotes EGFR-mediated signaling (FIG. 8). In this capacity, previous work has shown that EGFR phosphorylates the MUC1-C cytoplasmic domain and that this domain binds to PI3K and contributes to the activation of PI3K->AKT signaling (FIG. 8) (Li et al., 2001; Raina et al., 2004; Ramasamy et al., 2007; Raina et al., 2011). Accordingly, MUC1-C silencing in EGF-stimulated MCF-10A cells attenuated activation of AKT and the induction of a proliferative response. These findings support a model in which EGF-induced MUC1-C translation activates an auto-inductive loop in which MUC1-C in turn contributes to EGFR signaling (FIG. 8).

MUC1-C Translation is Induced by the eIF4A RNA Helicase.

AKT controls translation in part through the activation of mTORC1, which results in the phosphorylation of several substrates, including S6K (FIG. 8) (Ma and Blenis, 2009). In turn, S6K phosphorylates and thereby induces the degradation of PDCD4, an inhibitor of eIF4A RNA helicase activity that regulates translation of proteins, such as p53, that are involved in growth and survival (FIG. 8) (Dorrello et al., 2006; Wedeken et al., 2011). S6K also phosphorylates eIF4B, which interacts with eIF4A and contributes to eIF4A activation (Parsyan et al., 2011). In studies with MCF-10A cells, EGF-induced increases in MUC1-C translation were blocked by silencing S6K1. Moreover, EGF stimulation was associated with downregulation of PDCD4, suggesting that the induction of MUC1-C translation could be mediated by the eIF4A RNA helicase. To directly address this possibility, the inventor found that EGF- and HRG-induced MUC1-C translation was substantially blocked by silvestrol, a natural product isolated from the plant Aglaia silvestris (Hwang et al., 2004). Silvestrol inhibits eIF4A by inducing dimerization of eIF4A and RNA (Bordeleau et al., 2008) and preferentially blocks the translation of mRNAs with highly structured 5′UTRs that require efficient unwinding by the eIF4F complex (Cencic et al., 2009). In that sense, translation of specific mRNAs varies substantially for different transcripts and is dependent in part on the presence of discrete hairpin structures in the 5′UTR (De Benedetti and Graff, 2004). Notably, the MUC1 5′UTR includes such discrete hairpin structures, consistent with a potential requirement for unwinding by the eIF4A RNA helicase for translation initiation (FIG. 8). In concert with this model, silvestrol blocked growth factor-induced MUC1-C translation, but had little effect on the abundance of β-actin, which is encoded by a mRNA with a relatively unstructured 5′UTR (De Benedetti and Graff, 2004). To confirm these results, the inventor showed that CR-1-31-B, a novel inhibitor of the eIF4A RNA helicase (Rodrigo et al., 2012), similarly blocks MUC1-C translation in response to growth factor stimulation. By contrast and as a control for specificity, an inactive enantiomer of CR-1-31-B, designated CR-1-30-B, had no apparent effect on the induction of MUC1-C translation. These findings indicate that, like certain other oncoproteins (De Benedetti and Graff, 2004), the translation of MUC1-C is preferentially induced by growth factor stimulation and activation of the eIF4A RNA helicase.

Targeting Cap-Dependent Translation to Block Overexpression of MUC1-C in Human Cancers.

Dysregulation of protein synthesis has been linked to the development and progression of cancers as a result of aberrant cell signaling pathways that converge on translation initiation (Blagden and Willis, 2011). For that reason, drugs have been developed to inhibit mRNA translation by blocking eIF4E, eIF4A and other targets that are components of the translational machinery (Blagden and Willis, 2011). MUC1-C is aberrantly overexpressed in breast and other human cancers, and thereby contributes to growth and survival pathways (Kufe, 2009). Thus, MUC1-C has become an attractive target for the treatment of cancers that overexpress this oncogenic subunit (Kufe, 2009). The present results indicate that targeting MUC1-C translation represents a potential approach to inhibit the effects of MUC1-C overexpression in cancer cells. In the breast cancer cells studied in the present work, eIF4E levels were similar to those found in non-malignant MCF-10A cells. Notably, however, PDCD4 expression was decreased compared to that in MCF-10A cells, suggesting that the eIF4A RNA helicase could be of importance to the increased levels of MUC1-C in breast cancer cells. Notably, in concert with the findings in growth factor-stimulated MCF-10A cells, treatment of breast cancer cells with silvestrol was associated with decreases in MUC1-C abundance. Treatment with CR-1-31-B, but not CR-1-30-B, also resulted in downregulation of MUC1-C levels, indicating that the eIF4A RNA helicase activity is responsible, at least in part, for overexpression of MUC1-C in these cells. Cell-penetrating peptide and small molecule inhibitors have been developed that directly block the MUC1-C oncogenic function and induce death of breast cancer cells (Raina et al., 2009; Zhou et al., 2011). Targeting MUC1-C translation to decrease MUC1-C abundance could thus conceivably increase the effectiveness of these direct inhibitors. Finally, agents such as silvestrol and CR-1-31-B, are likely to be highly effective as anti-cancer agents given that, in addition to MUC1-C, multiple oncoproteins, including cyclin D1, MYC and MCL1, are downregulated by inhibiting the eIF4A RNA helicase. Indeed, preclinical studies with silvestrol in animal models have demonstrated promising anti-tumor activity with little toxicity, supporting the selectivity of blocking eIF4A function for cancer treatment (Bordeleau et al., 2008; Lucas et al., 2009; Schatz et al., 2011).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference:
U.S. Pat. No. 4,415,723
U.S. Pat. No. 4,458,066
U.S. Pat. No. 5,354,855
U.S. Pat. No. 5,440,013
U.S. Pat. No. 5,446,128
U.S. Pat. No. 5,475,085
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,795,715
U.S. Pat. No. 5,840,833
U.S. Pat. No. 5,859,184
U.S. Pat. No. 5,889,136
U.S. Pat. No. 5,929,237
U.S. Pat. No. 6,261,569
U.S. Pat. No. 6,420,393
U.S. Pat. No. 6,710,075
U.S. Pat. No. 6,943,182
U.S. Pat. No. 7,183,059
U.S. Pat. No. 7,192,713
U.S. Patent Appln. 2005/02506890
U.S. Patent Appln. 2006/0008848
Ahmad et al., Cancer Res., 69:7013-7021, 2009.
Ahmad et al., Science Signaling, 4(160):ra9, 2011.
Baldus et al., Clin. Cancer Res., 10(8):2790-2796, 2004.
Blagden and Willis, Nat. Rev. Clin. Oncol., 8:280-291, 2011.
Bodanszky et al., J. Antibiot., 29(5):549-53, 1976.
Bordeleau et al., J. Clin. Invest., 118:2651-2660, 2008.
Bosher and Labouesse, Nat. Cell. Biol., 2(2):E31-E36, 2000.
Caplen et al., Gene, 252(1-2):95-105, 2000.
Carruthers et al., Proc. Natl. Acad. Sci. USA, 97(24):13080-13085, 2000.
Cencic et al., PLoS One, 4:e5223, 2009.
Cook et al., Cell, 27:487-496, 1981.
De Benedetti and Graff, Oncogene, 23:3189-3199, 2004.
Dorrello et al., Science, 314:467-471, 2006.
Elbashir et al., Nature, 411(6836):494-498, 2001.
Fire et al., Nature, 391(6669):806-811, 1998.
Fischer, Med. Res. Rev., 27(6):755-796, 2007.
Forster and Symons, Cell, 49(2):211-220, 1987.
Gendler et al., J. Biol. Chem., 263:12820-12823, 1988.
Gerlach et al., Nature (London), 328:802-805, 1987.
Grishok et al., Science, 287:2494-2497, 2000.
Hsieh et al., Br. J. Cancer, 105:329-336, 2011.
Huang et al., Cancer Biol Ther., 2:702-706, 2003.
Huang et al., Cancer Res., 65:10413-10422, 2005.
Hwang et al., J. Org. Chem., 69:3350-3358, 2004.
Jansen et al., Cancer Res., 65:6034-6041, 2005.
Johnson et al., In: Biotechnology And Pharmacy, Pezzuto et al. (Eds.), Chapman and Hall, NY, 1993.
Joshi et al., Mol. Cancer Ther., 8:3056-3065, 2009.
Joyce, Nature, 338:217-244, 1989.
Ketting et al., Cell, 99(2):133-141, 1999.
Kim and Cook, Proc. Natl. Acad. Sci. USA, 84(24):8788-8792, 1987.
Kinlough et al., J. Biol. Chem., 279(51):53071-53077, 2004.
Kong and Yamori, Curr. Med. Chem., 16:2839-2854, 2009.
Kufe et al., Hybridoma, 3:223-232, 1984.
Kufe, Nat. Rev. Cancer, 9:874-885, 2009.
Levitan et al., J. Biol. Chem., 280:33374-33386, 2005.
Li et al., J. Neurosci. 24:4070-4081, 2004.
Li et al., Oncogene, 22:6107-6110, 2003a.
Li et al., Cancer Biol. Ther., 2:187-193, 2003b.
Li et al., Mol. Cancer Res., 1:765-775, 2003c.
Li et al., J. Biol. Chem., 276:35239-35242, 2001a.
Li et al., J. Biol. Chem., 276:6061-6064, 200 lb.
Li et al., Oncogene, 22:6107-6110, 2003.
Li et al., Mol. Cell Biol., 18:7216-7224, 1998.
Ligtenberg et al., J. Biol. Chem., 267, 6171-6177, 1992.
Lin and Avery, Nature, 402:128-129, 1999.
Lucas et al., Blood, 113:4656-4666, 2009.
Ma and Blenis, Nat. Rev. Mol. Cell Biol., 10:307-318, 2009.
Merlo et al., Cancer Res., 49, 6966-6971, 1989.
Merrifield, J. Am. Chem. Soc., 85:2149-2154, 1963.
Michel and Westhof, J. Mol. Biol., 216:585-610, 1990.
Montgomery et al., Proc. Natl. Acad. Sci. USA, 95:15502-15507, 1998.
Newton et al., Br. J. Pharmacol., 130:1353-1361, 2000.
Palamarchuk et al., Cancer Res., 65:11282-11286, 2005.
Parsyan et al., Nat. Rev. Mol. Cell Biol. 12:235-245, 2011
PCT Appln. WO 00/44914
PCT Appln. WO 01/36646
PCT Appln. WO 01/68836
PCT Appln. WO 08/121767
PCT Appln. WO 99/32619
Peptide Synthesis, 1985
Perey et al., Cancer Res., 52(22):6365-6370, 1992.
Pochampalli et al., Oncogene, 26:1693-1701, 2007.
Protective Groups in Organic Chemistry, 1973
Raina et al., Cancer Res., 69:5133-5141, 2009.
Raina et al., J. Biol. Chem., 279:20607-20612, 2004.
Raina et al., Mol. Cancer Therapeutics, 10:806-816, 2011.
Ramasamy et al., Mol. Cell, 27:992-1004, 2007.
Reinhold-Hurek and Shub, Nature, 357:173-176, 1992.
Remington's Pharmaceutical Sciences, 15^{th} Ed., 1035-1038 and 1570-1580, 1990.
Remington's Pharmaceutical Sciences, 15^{th} Ed., 33:624-652, 1990.
Ren et al., Cancer Cell, 5:163-175, 2004.
Ren et al., J. Biol. Chem., 277:17616-17622, 2002.
Rodrigo et al., J. Med. Chem., 55(1):558-562, 2012.
Rogers et al., Frog. Nucleic Acid Res. Mol. Biol., 72:307-331, 2002.
Sarver et al., Science, 247:1222-1225, 1990.
Scanlon et al., Proc. Natl. Acad. Sci. USA, 88:10591-10595, 1991.
Schafmeister et al., J. Amer. Chem. Soc., 122(24): 5891-5892, 2000.
Schatz et al., J. Exp. Med., 208:1799-1807, 2011.
Schroeder et al., J. Biol. Chem., 276(16):13057-13064, 2001.
Sharp and Zamore, Science, 287:2431-2433, 2000.
Sharp, Genes Dev., 13:139-141, 1999.
Siddiqui et al., Proc. Natl. Acad. Sci. USA, 85:2320-2323, 1988.
Solid Phase Peptide Synthelia, 1984
Sonenberg et al., Cell, 136:731-745, 2009.
Tabara et al., Cell, 99(2):123-132, 1999.
Truscott et al., J Cell Biol., 163(4):707-713, 2003.
Vermeer et al., Nature, 422:322-326, 2003.
Walensky et al., Science 305:1466-1470, 2004.
Wedeken et al., J. Biol. Chem. 286:42855-862, 2011.
Wei et al., Cancer Cell, 7:167-178, 2005.
Weinberg, Ciba Found Symp., 142:99-105, 1989.
Wen et al., J. Biol. Chem., 278:38029-38039, 2003.
Wendel et al., Genes Dev., 21:3232-3237, 2007.
Wincott et al., Nucleic Acids Res., 23(14):2677-2684, 1995.
Workman et al., Cancer Res., 70:2146-2157, 2010.
Yamamoto et al., J. Biol. Chem., 272:12492-12494, 1997.
Yin and Kufe, J. Biol. Chem., 278:35458-35464, 2003.
Yin et al., J. Biol. Chem., 282:257-266, 2007.
Young et al., Cell. 112(1):41-50, 2003.
Zhou et al., Mol. Pharm., 79:886-893, 2011.

1. A method of inhibiting a cancer cell that expresses MUC1 comprising contacting said cancer cell with an inhibitor of eIF4A RNA helicase.

2. The method of claim 1, wherein said inhibitor is an inhibitor of eIF4A RNA helicase expression.

3. The method of claim 1, wherein said inhibitor is an inhibitor of eIF4A RNA helicase activity.

4. The method of claim 3, wherein said inhibitor is silvestrol or an analog thereof.

5. The method of claim 1, wherein said cancer cell is metastatic, recurrent or multidrug resistant cancer cell.

6. (canceled)

7. The method of claim 1, wherein said cancer cell is a carcinoma cell, a leukemia cell or a myeloma cell.

8. The method of claim 7, wherein the carcinoma cell is a prostate or breast carcinoma cell.

9. The method of claim 1, further comprising contacting said cancer cell with a MUC1 peptide of at least 4 consecutive MUC1 residues and no more than 20 consecutive MUC1 residues and comprising the sequence CQC (SEQ ID NO:4), wherein the amino-terminal cysteine of CQC is covered on its NH₂-terminus by at least one amino acid residue that need not correspond to the native MUC1 transmembrane sequence.

10-15. (canceled)

16. A method of treating MUC1-expressing cancer in a subject comprising administering to said subject an inhibitor of eIF4A RNA helicase.

17. The method of claim 16, wherein said inhibitor is an inhibitor of eIF4A RNA helicase expression.

18. The method of claim 16, wherein said inhibitor is an inhibitor of eIF4A RNA helicase activity.

19. The method of claim 18, wherein said inhibitor is silvestrol or an analog thereof.

20. The method of claim 16, wherein said cancer is metastatic, recurrent or multidrug resistant.

21. (canceled)

22. The method of claim 16, wherein said cancer is a carcinoma, a leukemia or a myeloma.

23. The method of claim 22, wherein the carcinoma is a prostate or breast carcinoma.

24. The method of claim 16, further comprising administering to said subject a second anti-cancer therapy.

25. The method of claim 24, wherein said second anti-cancer therapy is surgery, chemotherapy, radiotherapy, hormonal therapy, toxin therapy, immunotherapy, cryotherapy, a MUC1 ligand TRAP, or a small molecule inhibiting MUC1 dimer formation.

26-28. (canceled)

29. The method of claim 24, wherein said second anti-cancer therapy comprises administering to said subject a MUC1 peptide of at least 4 consecutive MUC1 residues and no more than 20 consecutive MUC1 residues and comprising the sequence CQC (SEQ ID NO:4), wherein the amino-terminal cysteine of CQC is covered on its NH₂-terminus by at least one amino acid residue that need not correspond to the native MUC1 transmembrane sequence.

30-35. (canceled)

36. The method of claim 16, wherein administering comprises intravenous, intra-arterial, intra-tumoral, subcutaneous, topical or intraperitoneal administration.

37. The method of claim 16, wherein administering comprises local, regional (e.g., into tumor vasculature), systemic, or continual administration.

38. (canceled)

39. The method of claim 16, wherein said subject is a human.

40-45. (canceled)

46. The method of claim 16, further comprising, prior to administering, the step of assessing MUC1 expression in a cancer cell from said subject.

47-48. (canceled)

49. A kit comprising (a) a MUC1 detection agent and (b) and eIF4A inhibitor.

50. (canceled)


SEQUENCE LISTING (55 sequences)

SEQ ID NO: 1 - protein, 159 aa, Homo sapiens
GSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 2 - protein, 72 aa, Homo sapiens
CQCRRKNYGQLDIFPARDTYHPMSEYPTYHTHGRYVPPSSTDRSPYEKVSAGNGGSSLSYTNPAVAATSANL

SEQ ID NO: 3 - protein, 15 aa, Homo sapiens
CQCRRKNYGQLDIFP

SEQ ID NO: 4 - protein, 6 aa, Artificial Sequence
  Synthetic peptide
CQCRRK

SEQ ID NO: 5 - protein, 34 aa, Artificial Sequence
  Synthetic peptide
QAATATRGRSAASRPTERPRAPARSASRPRRPVE

SEQ ID NO: 6 - protein, 16 aa, Artificial Sequence
  Synthetic peptide
RQIKIWFQNRRMKWKK

SEQ ID NO: 7 - protein, 7 aa, Artificial Sequence
  Synthetic peptide
RRMKWKK

SEQ ID NO: 8 - protein, 16 aa, Artificial Sequence
  Synthetic peptide
RRWRRWWRRWWRRWRR

SEQ ID NO: 9 - protein, 18 aa, Artificial Sequence
  Synthetic peptide
RGGRLSYSRRRFSTSTGR

SEQ ID NO: 10 - protein, 11 aa, Artificial Sequence
  Synthetic peptide
YGRKKRRQRRR

SEQ ID NO: 11 - protein, 9 aa, Artificial Sequence
  Synthetic peptide
RKKRRQRRR

SEQ ID NO: 12 - protein, 11 aa, Artificial Sequence
  Synthetic peptide
YARAAARQARA

SEQ ID NO: 13 - protein, 8 aa, Artificial Sequence
  Synthetic peptide
RRRRRRRR

SEQ ID NO: 14 - protein, 8 aa, Artificial Sequence
  Synthetic peptide
KKKKKKKK

SEQ ID NO: 15 - protein, 27 aa, Artificial Sequence
  Synthetic peptide
GWTLNSAGYLLGKINLKALAALAKXIL

SEQ ID NO: 16 - protein, 18 aa, Artificial Sequence
  Synthetic peptide
LLILLRRRIRKQANAHSK

SEQ ID NO: 17 - protein, 16 aa, Artificial Sequence
  Synthetic peptide
SRRHHCRSKAKRSRHH

SEQ ID NO: 18 - protein, 11 aa, Artificial Sequence
  Synthetic peptide
NRARRNRRRVR

SEQ ID NO: 19 - protein, 15 aa, Artificial Sequence
  Synthetic peptide
RQLRIAGRRLRGRSR

SEQ ID NO: 20 - protein, 13 aa, Artificial Sequence
  Synthetic peptide
KLIKGRTPIKFGK

SEQ ID NO: 21 - protein, 10 aa, Artificial Sequence
  Synthetic peptide
RRIPNRRPRR

SEQ ID NO: 22 - protein, 18 aa, Artificial Sequence
  Synthetic peptide
KLALKLALKALKAALKLA

SEQ ID NO: 23 - protein, 14 aa, Artificial Sequence
  Synthetic peptide
KLAKLAKKLAKLAK

SEQ ID NO: 24 - protein, 27 aa, Artificial Sequence
  Synthetic peptide
GALFLGFLGAAGSTNGAWSQPKKKRKV

SEQ ID NO: 25 - protein, 21 aa, Artificial Sequence
  Synthetic peptide
KETWWETWWTEWSQPKKKRKV

SEQ ID NO: 26 - protein, 23 aa, Artificial Sequence
  Synthetic peptide
GALFLGWLGAAGSTMGAKKKRKV

SEQ ID NO: 27 - protein, 23 aa, Artificial Sequence
  Synthetic peptide
MGLGLHLLVLAAALQGAKSKRKV

SEQ ID NO: 28 - protein, 26 aa, Artificial Sequence
  Synthetic peptide
AAVALLPAVLLALLAPAAANYKKPKL

SEQ ID NO: 29 - protein, 28 aa, Artificial Sequence
  Synthetic peptide
MANLGYWLLALFVTMWTDVGLCKKRPKP

SEQ ID NO: 30 - protein, 24 aa, Artificial Sequence
  Synthetic peptide
LGTYTQDFNKFHTFPQTAIGVGAP

SEQ ID NO: 31 - protein, 26 aa, Artificial Sequence
  Synthetic peptide
DPKGDPKGVTVTVTVTVTGKGDPXPD

SEQ ID NO: 32 - protein, 14 aa, Artificial Sequence
  Synthetic peptide
PPPPPPPPPPPPPP

SEQ ID NO: 33 - protein, 18 aa, Artificial Sequence
  Synthetic peptide
VRLPPPVRLPPPVRLPPP

SEQ ID NO: 34 - protein, 10 aa, Artificial Sequence
  Synthetic peptide
PRPLPPPRPG

SEQ ID NO: 35 - protein, 30 aa, Artificial Sequence
  Synthetic peptide
SVRRRPRPPYLPRPRPPPFFPPRLPPRIPP

SEQ ID NO: 36 - protein, 21 aa, Artificial Sequence
  Synthetic peptide
TRSSRAGLQFPVGRVHRLLRK

SEQ ID NO: 37 - protein, 23 aa, Artificial Sequence
  Synthetic peptide
GIGKFLHSAKKFGKAFVGEIMNS

SEQ ID NO: 38 - protein, 37 aa, Artificial Sequence
  Synthetic peptide
KWKLFKKIEKVGQNIRDGIIKAGPAVAVVGQATQIAK

SEQ ID NO: 39 - protein, 28 aa, Artificial Sequence
  Synthetic peptide
ALWMTLLKKVLKAAAKAALNAVLVGANA

SEQ ID NO: 40 - protein, 26 aa, Artificial Sequence
  Synthetic peptide
GIGAVLKVLTTGLPALISWIKRKRQQ

SEQ ID NO: 41 - protein, 14 aa, Artificial Sequence
  Synthetic peptide
INLKALAALAKKIL

SEQ ID NO: 42 - protein, 33 aa, Artificial Sequence
  Synthetic peptide
GFFALIPKIISSPLPKTLLSAVGSALGGSGGQE

SEQ ID NO: 43 - protein, 15 aa, Artificial Sequence
  Synthetic peptide
LAKWALKQGFAKLKS

SEQ ID NO: 44 - protein, 27 aa, Artificial Sequence
  Synthetic peptide
SMAQDIISTIGDLVKWIIQTVNXFTKK

SEQ ID NO: 45 - protein, 41 aa, Artificial Sequence
  Synthetic peptide
LLGDFFRKSKEKIGKEFKRIVQRIKQRIKDFLANLVPRTES

SEQ ID NO: 46 - protein, 20 aa, Artificial Sequence
  Synthetic peptide
LKKLLKKLLKKLLKKLLKKL

SEQ ID NO: 47 - protein, 18 aa, Artificial Sequence
  Synthetic peptide
KLKLKLKLKLKLKLKLKL

SEQ ID NO: 48 - protein, 18 aa, Artificial Sequence
  Synthetic peptide
PAWRKAFRWAWRMLKKAA

SEQ ID NO: 49 - protein, 10 aa, Artificial Sequence
  Synthetic peptide
GGGGSGGGGS

SEQ ID NO: 50 - protein, 15 aa, Artificial Sequence
  Synthetic peptide
GGGGSGGGGSGGGGS

SEQ ID NO: 51 - protein, 20 aa, Artificial Sequence
  Synthetic peptide
GGGGSGGGGSGGGGSGGGGS

SEQ ID NO: 52 - protein, 25 aa, Artificial Sequence
  Synthetic peptide
GGGGSGGGGSGGGGSGGGGSGGGGS

SEQ ID NO: 53 - protein, 10 aa, Artificial Sequence
  Synthetic peptide
EAAAKEAAAK

SEQ ID NO: 54 - protein, 15 aa, Artificial Sequence
  Synthetic peptide
EAAAKEAAAKEAAAK

SEQ ID NO: 55 - protein, 20 aa, Artificial Sequence
  Synthetic peptide
EAAAKEAAAKEAAAKEAAAK